(19) United States
(12) Patent Application Publication
(10) Pub. No.: US 2015/0238573 A1
(43) Pub. Date: Aug. 27, 2015

(54) NUTRITIONAL FOOD AND FEED, COMPOSITION, PROCESSING AND METHOD OF USE

(71) Applicant: Nutrinia Ltd., Nazareth (IL)

(72) Inventors: Amir Barzilay, Kochav Yair (IL); Hagit Koren-Lichtig, Moshav Mishmeret (IL); Shay Arendt-Cohen, Kibbutz Ramot Menashe-Doar-Na Galil Elyo (IL); Ezra Eilat, Tzur-Moshe (IL); Yael Gilutz, Tel-Aviv (IL); Aviv Hanien, Haifa (IL); Pierre Sharvit, Gan Yeoshia - Emek Hefer (IL); Lora Eshkar, Rishon-LeZion (IL); Naim Shehadeh, Kfar-Yassif (IL); Raanan Shamir, Herzlia (IL)

(21) Appl. No.: 14/705,816

(22) Filed: May 6, 2015

Related U.S. Application Data
(63) Continuation of application No. 11/597,664, filed on Nov. 24, 2006, now abandoned.
(60) Provisional application No. 60/627,154, filed on Nov. 15, 2004.
(60) Provisional application No. 60/603,570, filed on Aug. 24, 2004.
(60) Provisional application No. 60/573,376, filed on May 24, 2004.

(51) Int. Cl. A61K 38/28; A23K 1/18; A61K 9/50; A23L 1/302; A23L 1/29; A23L 1/305; A23K 1/16; A23L 1/00

(52) U.S. Cl. CPC A61K 38/28; A23K 1/1603; A23K 1/1631; A23K 1/1893; A23L 1/0029; A23L 1/302; A23L 1/296; A23L 1/305; A61K 9/5089; A61K 9/5073; A23V 2002/00

(57) ABSTRACT

The present invention relates to means for protecting and incorporating bioactive compounds in food or feed formulations used to enhance the health status and growth performance of human and non-human organisms.

### FIELD OF THE INVENTION

This invention relates generally to naturally found health and growth promoting compounds and its derivatives, their incorporation and delivery into nutritional food and feed and their application in supplementing the diet of human and non-human organisms.

### BACKGROUND OF THE INVENTION

Bioactive compounds, naturally present in unprocessed milk and eggs have been shown to have a positive effect on developmental, immunological, and nutritional aspects in several human and commercially viable livestock.

External sources of such compounds are usually obtained through the food chain, or alternatively, by way of feed. Most industrial food or feed production processes, involve manufacturing conditions that are destructive to the viability of bioactive compounds. In addition, supply chain constraints impose longer shelf life requirements where extended storage under adverse conditions, cause loss of efficacy of the biological activity of such compounds, making the unadulterated use of these compounds impractical, as well as often impossible. Bioactive compounds extracted from plants, recombinant organisms or otherwise artificially generated, may be produced at a lower cost, be free of bacteria and viruses frequently found in traditional sources, and be better accepted as healthier and safer by both regulatory authorities and the general public.

Since human neonates and newborn animal infants, are frequently weaned of their natural food or feed immediately or shortly after birth and are nourished primarily with artificially produced food or feed substitutes, the desired positive health and growth promoting benefits provided by the original natural food or feed are largely absent.

In addition, escalating energy and commodity costs, make it extremely challenging for livestock breeders and growers alike to continue improving the cost/performance production ratio, thereby maximizing the commercial value of the livestock. Therefore, there is a need of maximizing and optimizing feed conversion ratios; maximizing and optimizing weight gain, reducing mortality rates, improving meat nutritional composition, and accelerating the healthy growth of newborn livestock.

Correspondingly, human infants have different nutritional needs than those of children and adults. They require more fat and less protein than adults. Breast milk contains high concentrations of fat-digesting enzymes and bioactive proteins that allow for highly efficient fat absorption. Full term babies who are not fed enough linoleic acid suffer from dermtatosis and growth failure. These conditions are easily reversed when linoleic acid is added to the infant's diet. Fatty acid deficiency in a breast-feeding infant is a hazard of long term low fat parental dieting.

Suckling in humans and mammals has multiple beneficial effects on infants' well being. Optimal nutritional requirements, immune protection against a wide range of infection related diseases and, since it contains active insulin molecules it protects the infant against the development of Type-1 diabetes, as well as promoting small intestine growth and development (4).

Type-1 diabetes, which is insulin dependent diabetes mellitus (IDDM), is the consequence of progressive autoimmune pancreatic β-cell destruction during an initially asymptomatic period that may extend many years. The etiology is multifactorial, with genetic and environmental factors contributing to the autoimmune destruction of the β-cells. Many studies show that type-I diabetes is related to cow's milk consumption and neonatal feeding practices. In the case-control studies, patients with type-I diabetes were more likely to have been breast-fed for less than 3 months and to have been exposed to cow's milk proteins before 3 months of age. Moreover, the immune system of patients with IDDM recognizes cow's milk proteins as evident from analysis done with antibodies thus indicating that bioactive compounds remain bioactive after digestion. These data emphasize the importance of diet and orally administered bio active proteins on the development of autoimmune diabetes. The level of active bovine insulin in infant formulas is negligible, due to the harsh conditions associated with their manufacture.

Therefore, there is a recognized need for, a nutritional feed composition that will answer the need for optimal nutrition to newborn humans and animals, capable of delivering biomaterials including insulin in a manner that will guarantee their viability, both to the target organism, as well as the supply chain of its manufacture and similar need is recognized in supplementing the nutritional formula of term and preterm human neonates

### SUMMARY OF THE INVENTION

In one embodiment the present invention provides a nutritional composition for a subject, comprising a bioactive compound, identical, similar or analogous to one found in a natural food or feed source, and a protective layer, wherein release of the bioactive compound into the subject is in another embodiment the result of an environmental event.

In another embodiment, the invention provides a method for identifying a plant-derived health promoting compound comprising the selection of a health promoting candidate molecule from an organism source, followed by analyzing plants' genomic databases, or in another embodiment phylogenic databases, or in another embodiment physico-chemical properties of said health promoting candidate compound, or in another embodiment biological properties of said health promoting candidate molecule, or in another embodiment combination thereof; and screening the results in for a plant-derived compound, which is analogous to said candidate molecule, wherein the candidate compound is found in the natural food source, thereby identifying a plant derived health promoting compound, analogous to one which is found in one embodiment, in the natural food source of a subject.

In one embodiment, the invention provides a method for preparing an encapsulated bioactive compound in a nutritional food formulae or nutritional feed formulae or drink, comprising mixing a bioactive compound with an appropriate encapsulating material forming a blend, then processing the blend formed to form a functionally multilayered protected dry blend, wherein the protective layer is specifically designed in another embodiment, to degrade as a response to change in an environmental trigger and then adding the dry blend to the nutritional food formula or nutritional feed formulae or drink, thereby preparing a multilayered encapsulated bioactive compound in a nutritional human food formulae or human drink or nutritional animal feed formulae or animal drink.

In one embodiment, the invention provides a method for supplementing a nutritional food formula or feed formulae or drink of a mammal, an avian or a chordata, comprising incorporating a nutritional composition for a subject, comprising a bioactive compound identical, similar or analogous to one found in a natural food source, and a protective layer, wherein release of the bioactive compound into the subject is in another embodiment the result of an environmental event, in said nutritional food formula or nutritional feed formula or drink, thereby supplementing said human food or human drink or animal feed or animal drink.

### BRIEF DESCRIPTION OF THE DRAWINGS

FIG. 1 shows a visual description of the coating

### DETAILED DESCRIPTION OF THE INVENTION

In one embodiment the present invention provides a nutritional composition for a subject, comprising a bioactive compound, identical, similar or analogous to one found in a natural food source, and a protective layer, wherein release of the bioactive compound into the subject is in another embodiment the result of an environmental event.

In one embodiment, the term “bioactive compound” refers to any therapeutic substance which possesses desirable therapeutic characteristics or any health promoting substance which possess desirable health promoting characteristics for application to the health improvement or growth promotion improvement or growth performance improvement or prevention of diseases or elimination of potential diseases or reducing the onset of diseases of the organism. These agents are in another embodiment anti metabolites, or antiproliferatives in another embodiment, or anticancer chemotherapeutic agents in another embodiment, or anti-inflammatory steroid or non-steroidal anti-inflammatory agents in another embodiment, or immunosuppressive agents in another embodiment, or growth hormone antagonists in another embodiment, or growth factors in another embodiment, or dopamine agonists in another embodiment, or radiotherapeutic agents in another embodiment, or polypeptides in another embodiment, or peptides in another embodiment, or proteins in another embodiment, or enzymes in another embodiment, or extracellular matrix components in another embodiment, or free radical scavengers in another embodiment, or chelators in another embodiment, or antioxidants in another embodiment, or anti polymerases in another embodiment, or antiviral agents in another embodiment, or photodynamic therapy agents in another embodiment or gene therapy agents in another embodiment. In one embodiment, bioactive compounds refer to those compounds that prevent or in another embodiment reduce the onset of autoimmune diseases.

In one embodiment, the term ‘bioactive compounds” refers to any non-nutrient bioactive molecules naturally present in milk and eggs, targeting health promotion, growth promotion, growth improvement, disease prevention, disease reduction or therapeutics of human and non-human organisms. In another embodiment, identical, or in another embodiment similar or in another embodiment analogous substances from plant, recombinant, or chemical synthesis origin are also considered bioactive compounds to be used in the methods and compositions of the invention. In one embodiment, bioactive compounds of the invention refer to metabolites, or in another embodiment derivatives of any of the compounds described herein, which poses identical, or in another embodiment, similar, or in another embodiment, analogous, or in another embodiment, completely different bioactive properties, resulting from enzymatic degradation in one embodiment or any other natural or natural-like cleavage process in another embodiment. In one embodiment such processes, when involving proteins are considered encompassed within the scope of the invention even when such degradation reduces the protein, polypeptide, peptide, hormone or enzyme to a derivative of no less than 3 amino acids.

In another embodiment, the natural foods of newborn human infants and the feeds of newborn animal infants is natural unprocessed milk or in another embodiment natural unprocessed eggs, contain a broad number of health promoting or growth enhancing compounds. In one embodiment, such compounds are: bioactive proteins, bioactive hormones, bioactive polypeptides and bioactive peptides, or in another embodiment EGF (Epidermal Growth Factor), or in another embodiment insulin and insulin-like growth factors, or in another embodiment insulin-like growth factors' binding proteins, or in another embodiment immunoglobulins (e.g. H. Pylori antibody), or in another embodiment proline-rich polypeptides, or in another embodiment lactoferrin, or in another embodiment proteases, or in another embodiment lactalbumin, or in another embodiment interleukin, or in another embodiment lysozyme, or in another embodiment TGFA (Transforming Growth Factor A) or in another embodiment PDGF (Platelet Derived Growth Factor).

In one embodiment, the term “growth enhancing” refers to compounds that improve growth rate of or tissue mass accumulation or accelerated proliferation of tissues in a subject, or in another embodiment improve weight gain in a subject, or in another embodiment improve the food or feed conversion ratio in a subject, or in another embodiment modify the body composition of a subject, such as in another embodiment, hormones.

In one embodiment the term “bioactive compound” or “bioactive agent” refers to compounds having a biological effect. In one embodiment bioactive compounds are pharmaceutical compounds, or antibodies in another embodiment, or receptor ligands in another embodiment, or viruses in another embodiment, or proteins in another embodiment, or protein fragments in another embodiment, or polypeptides in another embodiment, or polypeptide fragments in another embodiment, or peptides in another embodiment, or peptide fragments in another embodiment, or oligopeptides in another embodiment. In one embodiment protein metabolites or derivatives which maintain or posses biological activity are the bioactive compounds. In another embodiment, metabolism of the bioactive compounds is carried out ex-vivo and incorporated in the compositions and methods of the invention. In another embodiment, ex-vivo digestion of a bioactive protein or bioactive polypeptide or bioactive peptide or bioactive hormone is used, wherein in one embodiment, digestion is done with enzymes, or in another embodiment, with chemical methods known to the skilled practitioner, or in another embodiment, by physical methods known in the art.

In another embodiment, the bioactive compound is an analogue of insulin, or in another embodiment an IGF-I, or in another embodiment an IGF-2, or in another embodiment an EGF, or in another embodiment any functional derivatives thereof. In one embodiment, the term “functional derivative” of insulin in one embodiment, refers to the product of enzymatic digestion of insulin (e.g. by trypsin, chymotrypsin, lysine-C, or elastase), which in another embodiment, is carried out ex-vivo, wherein the products of the digestion are collected and added into the nutritional composition and methods described herein. In one embodiment, the term “functional derivative” refers to a metabolite or in another embodiment, a degradated byproduct of a bioactive protein or bioactive polypeptide or bioactive peptide or bioactive hormone which still possesses bioactive properties and is identical, or in another embodiment similar, or in another embodiment analogous or in another embodiment completely different from the original molecule. In one embodiment “functional derivatives” refer to the ex-vivo treatment of the insulin in another embodiment, with Tris/HCl/1 mM 2-mercaptoethanol for a period of time followed by quenching using, in one embodiment phenylmethanesulfonyl fluoride.

In one embodiment, the natural food source is naturally unprocessed milk, or in another embodiment naturally unprocessed eggs, or in another embodiment plant material, or in another embodiment animal tissue or in another embodiment recombinant organism, or in another embodiment the result of PCR, or in another embodiment the result of chemical synthesis. In another embodiment, the natural food source of a subject for the purposes of this invention is a combination thereof.

In one embodiment, the term “subject” refers to any member of the mammal, avian or chordata phylum.

Plants show to naturally produce bioactive compounds that are in one embodiment analogous to its counterpart compounds in the natural foods and feeds of the organism, such as those found in one embodiment in naturally unprocessed milk or in another embodiment, naturally unprocessed eggs. In one embodiment, the term “bioactive molecule” refers to any molecule, e.g., protein, polypeptide, peptide, hormone, small organic molecule, carbohydrates (including polysaccharides), polynucleotide, lipids, etc. In another embodiment, a plurality of assay mixtures are run in parallel with different molecular concentrations to obtain a differential response to the various concentrations. In one embodiment, one of these concentrations serves as a negative control, i.e., at zero concentration or below the level of detection. In another embodiment, positive controls, i.e. the use of agents of known activity to alter or modulate the selected bioactive molecule activity, are used. In one embodiment, the terms “analogous” or “analog” or “analogue” interchangeably refer to a structure that is similar in function to one in another kind of organism but is of dissimilar evolutionary origin.

In another embodiment such compound is an insulin-like compound found in a number of plant varieties. While the amino acids sequence of such compound in plants may, in another embodiment be analogous to bovine insulin or in one embodiment, to insulin from another organism's origin, its structure is different. In one embodiment, the bioactivity of such insulin-like compound is similar to the bioactivity of a number of animal and human insulins. In another embodiment, such a compound serves as a substitute to such animal and human insulins.

Therefore, according to this aspect of the invention and in one embodiment, the invention provides a method to utilize plant-extracted bioactive compounds which are analogous to milk and eggs bioactive compounds, as supplements for human infant foods and animal infant feeds. In one embodiment, the mammal is a preterm human infant, or in another embodiment a term human infant. In one embodiment, the mammal is a human baby, human toddler, human adolescence, human adult or human old person. In one embodiment, the mammal animal or avian animal or chordata animal is a grown animal or mature animal.

The skilled person would recognize that in nature, the concentration of bioactive compounds (in milk or in eggs) is at nanograms/microgram levels, so when these compounds are supplemented in one embodiment to food or feed or drink at these levels, the health promoting or growth promoting properties of a bio active compound are on a physiological level, while when supplemented in much higher levels at another embodiment, the effect of the bioactive compound may be therapeutic.

In one embodiment, the bioactive compound is extracted from natural milk, or in another embodiment from natural eggs, or in another embodiment from animal tissue, or in another embodiment, harvested from recombinant DNA technology, or in another embodiment, extracted from plants or in another embodiment, synthetically produced.

In another embodiment, the term “Recombinant DNA” refers to a nucleic acid which is not naturally occurring, or which is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. In one embodiment, it is performed to join together nucleic acid segments of desired functions to generate a desired combination of functions, which in another embodiment is used to generate the function of the desired health promoting or growth promoting or disease eliminating or disease reducing bioactive compounds used in the compositions and methods of the invention.

In one embodiment, the release of the bioactive compound or in another embodiment, the bioactive compound derivative, into the nutritional composition of the invention, or in another embodiment, directly to the subject receiving the nutritional compositions of the invention, is following exposure to an environmental trigger. In another embodiment, the term “trigger” refers to a change in environmental conditions sufficient to initiate degradation in the the encapsulating materials of the encapsulating layers used in the composition and methods of the invention, the change leading to release of the bioactive, viable compounds encapsulated therein. In one embodiment, the reference environmental condition is time, or in another embodiment temperature, or in another embodiment moisture content, or in another embodiment pressure, or in another embodiment pH, or in another embodiment ionic strength, or in another embodiment enzymatic activity, or in another embodiment a combination thereof.

In one embodiment the environmental condition change may be by a change of ±2.5% in the reference environmental condition, or in another embodiment a change of ±5% in the reference environmental condition, or in another embodiment a change of ±10% in the reference environmental condition, or in another embodiment a change of ±15% in the reference environmental condition, or in another embodiment a change of ±20% in the reference environmental condition, or in another embodiment a change of ±25% in the reference environmental condition, or in another embodiment a change of ±30% in the reference environmental condition, or in another embodiment a change of ±35% in the reference environmental condition, or in another embodiment a change of ±40% in the reference environmental condition, or in another embodiment a change of ±45% in the reference environmental condition, or in another embodiment a change of ±50% in the reference environmental condition, or in another embodiment by a change of more than ±50% in the reference environmental condition.

In one embodiment, a protective layer surrounding or incorporating a bioactive compound is specifically designed to degrade, or in another embodiment, undergo controlled release, as a response to exposure to the change in environmental condition, which is in another embodiment time, or in another embodiment temperature, or in another embodiment moisture content, or in another embodiment pressure, or in another embodiment pH, or in another embodiment ionic strength, or in another embodiment enzymatic activity, or in another embodiment a combination thereof.

Therefore, according to this aspect of the invention and in one embodiment, a core wherein an active compound is embedded, is coated with an encapsulating wall material that will degrade rapidly when exposed to increased moisture, while protecting the active compound of the core when exposed to high temperature, such as in another embodiment, those encountered during pelleting processing, or extrusion processing, or baking process in another embodiment, or direct steam injection in another embodiment, or storage conditions imposing high temperatures or moisture or any combination thereof. In another embodiment, the core used with the methods and compositions of the invention encapsulates the bioactive material, which in another embodiment, is IGF-I, or IGF-II or EGF in another embodiment, or insulin in another embodiment, or functional fragment thereof. In one embodiment the active core as described hereinabove is further encapsulated in a material designed to protect the active core from digestion in a digestive system of a subject, and release the core which in another embodiment, releases the active compound, only as a response to an increase in pH.

In one embodiment the active core as described hereinabove is further encapsulated in a material designed to protect the active core from high temperatures, by enabling encapsulating material to absorb both high temperature and steam or moisture, and even partially degrade or melt, and by such heat and moisture absorption during partial or full degradation, protect the core and encapsulating layers inner to it. In another embodiment, the active core, which is encapsulated in an encapsulating material allowing release of the core based on increase in pH, is further encapsulated with another encapsulating material, designed to protect the core from increased temperature as described herein. The skilled artisan in the art, would recognize that the order of environmental triggers releasing the active compound is not rigid and depending on the environmental conditions of manufacturing, environmental conditions of integration into food or feed products, environmental conditions of storage after integration onto food or feed products, desired delivery location within the gastrointestinal system, timing and physiological activity desired, the encapsulating layers could accommodate those requirements without departing from the scope of the invention as described herein.

In one embodiment, any factor, which may affect the entrapment of the subject bioactive compound in a biodegradable matrix, and thereby affect its initial loading, in one embodiment, or, in another embodiment, subsequent release, or in another embodiment, a combination thereof, may be utilized according to the methods and compositions of this invention. In other embodiments, such factors may comprise inter-alia, the initial solvent concentration, its molecular size and polarity, the temperature and pressure under which the solvent is removed, molecular weight number (MWn) average of the biodegradable matrix, its polydispersity index, the size and polarity of the bioactive compound, when the biodegradable matrix is in another embodiment a polymer, the monomer ratio and distribution along the copolymer's chain, or a combination thereof. In addition, D/L ratio within each monomer of a biodegradable polymer will affect release rates. In one embodiment, the term D/L ratio refers to the ratio of monomer molecules that affect the direction (D-right, L-left), in which a cross-polarized lense will be rotated when observing a single optically active monomer, like lactic acid. Since most mammals have D-specific enzymes, that ratio will affect the digestion rate of the biodegradable biopolymer, affecting its molecular weight and consequently its viscosity, thereby affecting release rate of any entrapped bioactive compound.

In one embodiment, complexes between the bioactive molecule and the protective layer may be formed via covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. In one embodiment, modifications may increase the compound's solubility in aqueous solution, eliminate aggregation, enhance the physical and chemical stability of the compound, reduce the immunogenicity or reactivity of the compound or combination thereof.

In one embodiment, any of the compositions of this invention are used with any of the methods of the invention. In another embodiment, the compositions and methods of the invention are used in a nutritional supplement to human food or, in another embodiment, to animal feed.

In one embodiment of the invention a method is provided for identifying via a method, in a plant, a health promoting compound candidate and/or growth performance promoting compound which is identical to or sufficiently similar to or analogous to in its bioactive properties to a compound found in the natural food of human infants and/or found in the natural food or feed of animal infants, where such method includes a of, or a combination of: (i) analysis of genomic databases (ii) analysis of phylogenic databases (iii) chemical analysis instrumentation (iv) physical analysis instrumentation (v) biological analysis instrumentation (vi) bioactivity analysis instrumentation and methods.

According to this aspect of the invention, and in one embodiment, the invention provides a method for identifying a plant-derived health promoting or growth promoting compound comprising the selection of a health promoting candidate molecule from an organism source, followed by analyzing plants' genomic databases, or in another embodiment phylogenic databases, or in another embodiment physico-chemical properties of said health promoting or growth promoting candidate compound, or in another embodiment biological properties of said health promoting or growth promoting candidate molecule, or in another embodiment combination thereof; and screening the results in for a plant-derived compound, which is analogous to said candidate molecule, wherein the candidate compound is found in the natural food source, thereby identifying a plant derived health promoting or growth promoting compound, analogous to one which is found in one embodiment, in the natural food source of a subject.

In one embodiment candidate compounds encompass numerous chemical classes, though typically they are organic molecules, preferably small organic compounds having a molecular weight of more than about 100 daltons in one embodiment and less than about 100,000 daltons in another embodiment. Candidate molecules comprise in one embodiment functional groups necessary for structural interaction with proteins, particularly hydrogen bonding, and in another embodiment include at least an amine, carbonyl, hydroxyl or carboxyl group, or, in another embodiment at least two of the functional chemical groups. The candidate molecules comprise in one embodiment cyclical carbon or heterocyclic structures or in another embodiment, aromatic or polyaromatic structures substituted with one or more of the above functional groups. Candidate agents are found in one embodiment, among biomolecules including proteins, or in another embodiment polypeptides, or in another embodiment peptides, or in another embodiment hormones, or in another embodiment, saccharides, or in another embodiment, fatty acids, or in another embodiment, steroids, or in another embodiment, purines, or in another embodiment, pyrimidines, or in another embodiment, derivatives, or in another embodiment, structural analogs or combinations thereof. In one embodiment, the candidate molecule is a peptide.

In one embodiment bioactive compounds are present in plants in very low quantities, ranging from 1E-2 to 1E-12. Therefore, even when using the most sensitive instrumentation, discovery of the presence of many of such compounds is not trivial in plants. In order to succeed in identifying such compounds in plants, a method is provided to use at least two, but preferably more than two discovery methods, to identify the presence and yield of such compounds in plants. The method includes validation of the presence of the bioactive compound via genomic databases and phylogenic databases; and; identifying the presence of the compound via highly sensitive instrumentation such as HPLC in one embodiment, or GC-MS in another embodiment, or LC-MS in another embodiment; or MS-MS in another embodiment and in another embodiment, using quantitative analytical instrumentation such as ELISA and/or radioimmunoassay; and; using bioactivity kits, such as a cancer cells line or yeast which exclusively proliferates in the presence of a selected bioactive compound. Such complimentary combination of identification and and quantification methods is key to succeeding in the discovery of such low yield bioactive compounds in plants.

Candidate molecules are obtained in one embodiment from a wide variety of sources including libraries of synthetic or natural compounds. In another embodiment, numerous means are available for random and directed synthesis of a wide variety of organic compounds and biomolecules, including in one embodiment, expression of randomized oligonucleotides. In another embodiment, libraries of natural compounds in the form of bacterial, fungal, plant and animal extracts are available or in another embodiment, readily produced. In one embodiment, natural or synthetically produced libraries and compounds are readily modified through conventional chemical, physical and biochemical means. Known neutraceutical molecules may be subjected in one embodiment to directed or in another embodiment, to random chemical modifications, such as acylation, alkylation, esterification, amidification to produce structural analogs.

In one embodiment, the methods of this invention further comprise the steps of either analyzing the natural yield of the bioactive, plant-derived analog and, in another embodiment, increasing the yield of the bioactive compound. In another embodiment, the increase in yield of the plant-derived analog compound is carried out with methods known to one skilled in the art, such as in one embodiment, by selective breeding or in another embodiment, through genetic engineering methods, or any combination thereof. In one embodiment, the yield of the desired bioactive molecule to be used in the methods and compositions of this invention is increased in a plant and used.

In one embodiment, the candidate bioactive molecules are proteins. In another embodiment, the term “protein” refers to at least two covalently attached amino acids, which includes in one embodiment proteins, or in another embodiment polypeptides, or in another embodiment oligopeptides or in another embodiment peptides. The protein may be made up in one embodiment, of naturally occurring amino acids and peptide bonds, or in another embodiment, by synthetic peptidomimetic structures. In one embodiment, the terms “amino acid”, or “peptide residue”, refers to both naturally occurring and synthetic amino acids. In one embodiment, homo-phenylalanine, citrulline and norleucine are considered amino acids for the purposes of the invention. In another embodiment, the term “Amino acid” also includes imino acid residues such as proline in one embodiment and hydroxyproline in another embodiment. In one embodiment, the side chains may be in either the (R) or in another embodiment the (S) configuration or in another embodiment a racemic mixture thereof. If non-naturally occurring side chains are used in one embodiment, non-amino acid substituents may be used, for example to prevent or retard in vivo degradations. In another embodiment, chemical blocking groups or other chemical substituents are added.

In one embodiment, the candidate bioactive molecules are naturally occurring proteins or metabolites or fragments or derivatives of naturally occurring proteins. Thus, in another embodiment, cellular extracts containing proteins, or in another embodiment random or in another embodiment directed digests of proteinaceous cellular extracts, are used in the compositions and methods of the invention. In this way libraries of procaryotic and eukaryotic proteins may be made in one embodiment for screening in the systems described herein for a potential bioactive candidate molecule, to be used in another embodiment in the methods and compositions of the invention. In one embodiment, libraries of plant, bacterial, fungal, viral, chordate, avian, and mammalian proteins, are made.

In another embodiment, the candidate bioactive molecules are linked to a fusion partner. In one embodiment, the terms “fusion partner” or “functional group” refers to a sequence that is associated with the candidate bioactive molecule, that confers upon all members in that class a common function or ability. In another embodiment, fusion partners can be heterologous (i.e. not native to the host cell), or in one embodiment, synthetic (not native to any cell). Suitable fusion partners are in another embodiment, presentation structures, which provide the candidate bioactive molecules in a conformationally restricted or stable form; or in another embodiment targeting sequences, which allow the localization of the candidate bioactive molecule into a subcellular or extracellular compartment; or in another embodiment rescue sequences which allow the purification or isolation of either the candidate bioactive molecules or the nucleic acids encoding them; or in another embodiment stability sequences, which confer stability or protection from degradation to the candidate bioactive molecule or the nucleic acid encoding it, for example in one embodiment, resistance to proteolytic degradation; or in another embodiment dimerization sequences, to allow for peptide dimerization; or in another embodiment any combination thereof.

In another embodiment of the invention, a method is provided for extracting and analyzing the natural yield of a bioactive compound in a plant, where such method comprises: (i) chemical analysis instrumentation (iii) physical analysis instrumentation (iv) biological analysis instrumentation (v) bioactivity analysis instrumentation and methods.

A significant portion of the bioactive compounds in plants identical or similar or analogous in its bioactive properties to such compounds present in unprocessed milk and eggs, are proteins, peptides, polypeptides and hormones. The natural yield of all such compounds in the fruit part or any other organ of the plant is quite low and can range from 5% of the fresh organ weight to 0.01% of the fresh organ weight. Therefore, a first step in any extraction and purification method includes in one embodiment any extraction process which is capable of sufficiently isolating proteins, or in another embodiment peptides, polypeptides or in another embodiment hormones from the plant material, where such plant material can in one embodiment be fresh plant tissue, or dried plant tissue in another embodiment or grinded plant tissue in another embodiment. After such initial crude material extract, incorporating proteins, peptides, polypeptides or hormones is generated, there are a variety of processes to further purify a desired group of bioactive compounds, or a specific compound. For this purpose, the molecular weight of each bioactive compound may be used to cut off the undesired compounds, and leaving only the desired compound in a sufficient purification level.

In another embodiment of the invention, a method is provided for the improvement of the yield of such bioactive compound in a plant, where such yield improvement results from at one of, or a combination of, the following: (i) classical breeding of the plant (ii) genetic modification of the plant.

In order to find the highest natural yield in a specific plant or a family of plants, in one embodiment a significant list of different varieties of the plant or the plant family are cultivated. From the cultivated plants, samples are taken from all plant organs from very early growth stages in one embodiment, until very mature stages in another embodiment; In one embodiment each sample is extracted in different methods and analyzed in another embodiment, by multiple assay technologies. Thereby, in another embodiment a combination of plant variety, cultivation date, plant organ and extraction method exists, to find the highest natural yield. In one embodiment, improved plants or cell cultures from improved plants in another embodiment are used to grow the higher yielding plant material.

In order to make the method of using such naturally generated plant-borne compounds in a commercially viable manner, the natural yield of such compounds needs to be improved. Frequently, the yield needs to be improved in one or more orders of magnitude to make its extraction and utilization commercially viable. Such methods include, but not limited to, utilization of classical breeding, supported by genetic markers, to find specific plants with higher compound yields; and; utilization of genetic modification technologies in order to improve the expression of the target compounds in the desired plant variety. Such combination can improve the natural yield of the bioactive compound in the plant in one or more orders of magnitude.

In another embodiment of the present invention, a method is provided for extraction and purification of plant-based material, incorporating proteins, or in another embodiment peptides, or in another embodiment polypeptides, or in another embodiment hormones, with the purpose of generating in one embodiment a sufficiently purified compound with identical or similar or analogous bioactivity properties or health promoting activity properties or growth promoting activity properties to a compound found in the natural human infant food or newborn animal infant feed.

A significant portion of the bioactive compounds in plants is identical or similar or analogous in its bioactive properties to such compounds present in unprocessed milk and eggs, are proteins, peptides, polypeptides and hormones. The natural yield of all such compounds in any organ of the plant is quite low and can range from 5% of the fresh plant organ weight to 0.01% of the fresh plant organ weight. Therefore, a first step in any extraction and purification method may include any extraction process which is capable of sufficiently isolating proteins, peptides, polypeptides and hormones from the plant material, where such plant material can be fresh plant tissue, dried plant tissue or grinded plant tissue. After such initial crude material extract, incorporating proteins, peptides, polypeptides or hormones is generated, there are a variety of processes to further purify a desired group of bioactive compounds, or a specific compound. For this purpose, the molecular weight of each bioactive compound may be used to cut off the undesired compounds, and leaving only the desired compound in a sufficient purification level.

It is to be understood that any of the embodiments described hereinabove can be used with any of the methods embodiments of the invention.

In one embodiment, the invention provides a method for preparing an encapsulated bioactive compound in a nutritional food or feed, comprising mixing the bioactive compound with an appropriate encapsulating material forming a blend, then processing the blend formed to form a functionally multilayered protected dry blend, wherein the protective layer is specifically designed in another embodiment, to degrade as a response to change in an environmental trigger and then adding the dry blend to the nutritional food or feed, thereby preparing a multilayered encapsulation of the bioactive compound in a nutritional feed.

In another embodiment of the invention, a method is provided for the encapsulation, of a bio active compound, comprising; (i) mixing a bioactive compound with a wall-forming encapsulating material, and (ii) rapidly cooling the wall forming material thereby resulting in encapsulation of the bioactive compound. In one embodiment, the abovementioned process produces a core of a matrix entrapping the bioactive compound, where in another embodiment, the core does not initially contain a bioactive material and is therefore inert. In one embodiment, the core produced is substantially round, to improve the addition of additional encapsulating layers.

In one embodiment forming the round core further comprises flash freezing said liquid blend, collecting the droplets produced, lyophilizing the droplets collected and collecting the lyophilized droplets, thereby creating a round core, wherein said core may comprise a bioactive compound.

In the food and pharmaceutical industries, for example, microencapsulation is used to stabilize core materials, to control the timing and rate of the release of the core material and to isolate and prevent chemical interaction between reactive or incompatible components of a multicomponent formulation. Thus, in one embodiment, microencapsulation makes it possible to protect sensitive food or feed components, or in another embodiment, to ensure against nutritional value loss, or in another embodiment, to mask or preserve flavors and aromas. Encapsulation in one embodiment increases stability of vitamin supplements, for example, which are normally sensitive to electromagnetic radiation, both UV and visible, oxygen, metals, humidity and temperature. Microencapsulation is utilized in another embodiment to protect the lining of the mouth or the esophagus in one embodiment, from harsh, orally administered drugs which are released in the stomach by the action of stomach acids or stomach enzymes on the encapsulating wall material.

In one embodiment, encapsulation refers to the process where one or more bioactive compounds are coated with, or in another embodiment, entrapped within, another food grade or feed grade or pharma grade material or matrix. Encapsulation of heat sensitive compounds, such as for example nutraceutical components, enzymes or bioactive proteins, into matrixes that are edible and digestible, is generally difficult for a number of reasons. Conventional encapsulation processes, which expose matrix material and encapsulants to high temperatures and moisture such as those encountered in pelleting and extrusion, causes thermal destruction or loss of biological viability of the encapsulant. Thus, either large initial load of encapsulant, a very expensive and potentially hazardous preposition, would be required, or the encapsulant would not stand the encapsulation process at all. If the encapsulant can be encapsulated into a matrix under sufficiently low temperatures, the resulting product is a solid that is characterized as a hard glass-like solid that is capable of being processed further to yield a flowable powder, amenable to additional processing. In another embodiment, the temperature at which the particles are consumed, or in another embodiment, the eating temperature, is generally lower than 50 degrees Celsius, which is far below the glass transition temperature, Tg. Careful design of the glassy matrix can release the encapsulant containing the bioactive compound under desired conditions of temperature, moisture, pH or enzymatic environment. The encapsulated matrix could be used in one embodiment as dense pellets for a variety of processing applications, where a controlled release of the heat sensitive encapsulant is desired. The physical hardness of the products and their mechanical stability are advantageous in one embodiment for many processing applications.

In one embodiment, the encapsulant is food grade, or in another embodiment, feed grade. In one embodiment, the encapsulant is a polysaccharide, or in another embodiment a maltodextrin, or in another embodiment milk powder, or in another embodiment a whey protein, or in another embodiment a lipid, or in another embodiment a gum, or in another embodiment a cellulosics, or in another embodiment a amorphous lactose, or in another embodiment a combinations thereof.

In another embodiment, mixing the bioactive compound with an appropriate encapsulating material forming a blend further comprises mixing said compound with an encapsulant

In one embodiment, plasticizer as used herein means an additional compound capable of increasing the free volume of the liquid encapsulant without affecting the overall cumulative volume of both encapsulated matrix and the plasticizing compound.

In one embodiment of the invention, the invention provides a protected bioactive compound, including in one embodiment proteins for use in dietary formulations.

In another embodiment of the invention, the invention provides a method of manufacture of a protected bioactive compound to retain biological activity of these proteins.

In one embodiment, the invention may be used to preserve biological activity of a bioactive compound from adverse temperature, or in another embodiment, from adverse pressure, or in another embodiment, from adverse humidity, or in another embodiment, from adverse pH, or in another embodiment, from adverse osmotic concentration, or in another embodiment, from adverse ionic concentration, or in another embodiment, from adverse enzymatic degradation, or in another embodiment, from chemical degradation, or in another embodiment, presence of metals, or in another embodiment, surfactants and chelators, or in another embodiment, radiation (including in one embodiment UV, or IR, or Visible light or combination thereof), or in another embodiment, from microbial degradation. In another embodiment, the present invention may be used to protect bioactive compounds from physical changes including in one embodiment first or, in another embodiment second order phase transitions.

In one embodiment, the term “first order phase transition” refers to a discontinuity in the first derivative of Gibbs free energy with temperature at a constant concentration [(∂G/∂T)_{c}]. In another embodiment, the term “first order phase transition” refers to crystallization, or in another embodiment, to condensation, or in another embodiment, to evaporation, or in another embodiment, to melting.

In another embodiment, the term “second order phase transition” refers to a discontinuity in the second derivative of Gibbs free energy with temperature at a constant concentration [i.e (∂∂G/∂T)_{c}=(∂H/∂T)_{c}]. In another embodiment, the term “second order phase transition” refers to glass/rubber transition, or in another embodiment, to onset of rotational mobility (β-transition), or in another embodiment, to onset of vibrational mobility, or in another embodiment, to antemelting.

In one embodiment of the invention, a protected bioactive compound is provided, comprising a protecting layer enveloping a bioactive compound.

In another embodiment of the invention, an analogue to the protected bioactive compound is present in a natural mammalian milk or natural eggs, but its concentration is significantly lower, non viable, non available or non-existent in commercially processed human infant foods or animal infant feeds.

In one embodiment, “Mammal” for purposes of treatment refers to any animal classified as a mammal, including humans, domestic and farm animals, and zoo, sports, or pet animals, such as dogs, horses, cats, hamsters, rats, mice, cattle, pigs, goats, sheep, etc. In another embodiment, the mammal is human.

In another embodiment, concentration as used herein refers to Molar concentration and its fractions, or percentage relative to that existing in colostrum, full milk and eggs.

In one embodiment, the term “significantly lower” refers to the amount of the compound analogue to the bioactive compound in commercially processed milk is between about 0.01 to about 50 percent of that present in natural unprocessed colostrum, full milk or egg.

In one embodiment of the invention, the amount of the bioactive compound in commercially processed milk is no more than 50 percent of that present in natural unprocessed colostrum, full milk or egg.

In another embodiment of the invention, the amount of the bioactive compound in commercially processed milk is no more than, 25 percent of that present in natural unprocessed colostrum, full milk or egg.

In another embodiment of the invention, the amount of the bioactive compound in commercially processed milk is no more than 10 percent of that present in natural unprocessed colostrum, full milk or egg.

In another embodiment of the invention, the amount of the bioactive compound in commercially processed milk is no more than 1 percent of that present in natural unprocessed colostrum, full milk or egg.

In another embodiment of the invention, the amount of the bioactive compound in commercially processed milk is no more than 0.01 percent of that present in natural unprocessed colostrum, full milk or egg.

In one embodiment, the encapsulating material is food grade, or in another embodiment, the encapsulating material is feed grade, or in another embodiment, the encapsulating material pharma grade, or in another embodiment is a combination thereto.

In one embodiment, the invention provides a method for preparing at least one encapsulated bioactive compound in a nutritional food formulation or in a nutritional feed formulation, comprising mixing the bioactive compound with an appropriate encapsulating material forming a blend, then processing the blend formed to form a functionally multilayered protected dry blend, wherein each of the protective layers is specifically designed in another embodiment, to degrade as a response to change in an environmental trigger and then adding the dry blend to the nutritional food formulation or nutritional feed formulation, wherein the processing of the blend further comprises the forming of a round or non-round core, followed by drying of the core in a fluidized bed dryer, collecting the dehydrated core, suspending the dehydrated blend in a second functional encapsulating liquid, drying the suspension in a fluidized bed dryer and collecting the dehydrated suspension followed by resuspending the suspension obtained in the previous step in a third functional encapsulating fluid, then drying the resuspension a fluidized bed and finally adding the dry blend obtained to the nutritional food formulation or nutritional feed formulation, thereby preparing a multilayered encapsulation of a bioactive compound in a nutritional food formulation or a nutritional feed formulation. In one embodiment, the initial blend is liquid.

In one embodiment, a second protective layer, or in another embodiment a third protective layer, or in another embodiment a fourth protective layer, or in another embodiment a fifth protective layer, or in another embodiment a sixth protective layer, or in another embodiment a seventh protective layer, or in another embodiment an eighth protective layer, or in another embodiment a ninth protective layer, or in another embodiment a tenth protective layer further comprises a functional encapsulating material such as a maltodextrin, or a vitamin in another embodiment, or an antioxidant in another embodiment, or a protease inhibitor in another embodiment, or a growth hormone in another embodiment, or an EGF (Epidermal Growth Factor) in another embodiment, or an insulin and insulin-like growth factor in another embodiment, or an insulin-like growth factor's binding protein in another embodiment, or an immunoglobulin in another embodiment, or a proline-rich polypeptide in another embodiment, or a lactoferrin in another embodiment, or a protease in another embodiment, or a lactalbumin in another embodiment, or an interleukin in another embodiment, or a lysozyme in another embodiment, a TGFA (Transforming Growth Factor A) in another embodiment, or a PDGF (Platelet Derived Growth Factor) in another embodiment or combination thereof.

In one embodiment, the second, or in another embodiment the third functional encapsulating material or in another embodiment a fourth functional encapsulating material, or in another embodiment a fifth functional encapsulating material, or in another embodiment a sixth functional encapsulating material, or in another embodiment a seventh functional encapsulating material, or in another embodiment an eighth functional encapsulating material, or in another embodiment a ninth functional encapsulating material, or in another embodiment a tenth functional encapsulating material is maltodextrine, which, in another embodiment has a DE value between about 2 to about 64. In one embodiment the maltodextrine has a DE of between about 2 and about 5, or in another embodiment between about 5 and about 10, or in another embodiment between about 10 and about 15, or in another embodiment between about 15 and about 20, or in another embodiment between about 20 and about 25, or in another embodiment between about 25 and about 30, or in another embodiment between about 30 and about 35, or in another embodiment between about 35 and about 40, or in another embodiment between about 45 and about 50, or in another embodiment between about 50 and about 55, or in another embodiment between about 55 and about 60, or in another embodiment between about 60 and about 64. In one embodiment, the maltodextrine has a DE of 18. In another embodiment, the maltodextrine has a DE of 6.

In one embodiment of the invention, a protecting layer enables the maintenance of the bioactive properties of the bioactive compound while in a “dormant state”, which in one embodiment refers to the period when the protected bioactive compound is dehydrated, such as those present in powdered infant formulas, milk substitute products, and semi-solid/solid mixes and pellets. In another embodiment, the term “dormant state” of the bioactive compound refers to the preservation of the native tertiary and quaternary structures of the bioactive compound in an anhydrous state.

In one embodiment of the invention the protecting layer provides protection to the encapsulated bioactive compound, so that the bioactive compound shall maintain its bioactive properties in hostile conditions such as high temperatures normally leading in another embodiment to proteins' denaturation, or in another embodiment, high pressures, or in another embodiment, humidity, or in another embodiment, adverse osmotic pressures, or in another embodiment, high or low pH, or in another embodiment, strong enzymatic degradation, or in another embodiment, high solvent concentration and the like, or in another embodiment, a combination of at least two of the above. In another embodiment, based on a triggering event, an outer protection layer is dissolved, or in another embodiment outer protection layers are dissolved, and the “dormant” bioactive compound will be released and become physiologically active.

In one embodiment, the protected bioactive compound is designed in a way whereby the release of the bioactive compound occurs before entering the GI system of the human or animal consuming the formulation.

In another embodiment of the invention, the release may be while in contact with different parts of the gastrointestinal tract.

In one embodiment of the present invention, the encapsulated bioactive compound will be protected from conditions encountered during commercial pelleting and extrusion processes, including but not limited to cold pelleting and extrusion or hot pelleting extrusion either at standard temperatures and pressures or at conditions different than standard temperatures and pressures.

In another embodiment of the present invention, the encapsulated bioactive compound will be protected from conditions encountered during commercial size reduction processes, including in one embodiment colloid mills, both stator rotor of the frusto conical type, as well as crown and tooth type, or in another embodiment, ball mills, or in another embodiment, impact mills, or in another embodiment jet impingement mills, or in another embodiment, homogenizing mills, or in another embodiment, sonication, or in another embodiment, high velocity mixers and membrane emulsification devices.

In one embodiment of the present invention, the encapsulated bioactive compound will be protected from conditions encountered during commercial baking processes, or in another embodiment freezing processes.

In one embodiment, the external functional encapsulating material in the external encapsulating layer is designed to thermally protect the bioactive compound for no less than 2 minutes at a temperature of no less than 95° C. In another embodiment, the external functional encapsulating material in the external encapsulating layer is designed to thermally protect the bioactive compound for no less than 1 minutes at a temperature of no less than 120° C. In another embodiment, the external functional encapsulating material is designed to protect the bioactive compound from proteolitic enzymes and pH of no more than 4.75. In one embodiment, the external functional encapsulating material is designed to protect the bioactive compound from any combination of factors as described hereinabove.

In one embodiment, the invention provides a method for supplementing a nutritional food or feed or drink of a mammal, an avian or a chordata, comprising incorporating a nutritional composition for a subject, comprising a bioactive compound analogous to one found in a natural food source, and a protective layer, wherein release of the bioactive compound into the subject is in another embodiment the result of an environmental event, in said nutritional food or feed or drink, thereby supplementing said food or feed or drink.

Therefore, according to this aspect of the invention and in one embodiment, a newborn formulation is provided, comprising a bioactive compound being encapsulated or embedded in a multilayered edible ingredient, which protects and preserves the bioactive compound making it viable in the newborn.

It is noted, that during a period of several days prior to hatching, the pre-hatched avian chick is being partially fed through swallowing some of the amniotic fluid present in the pre-hatched fertilized egg. Further, it is also noted, that the in-ovo injection of a small volume of a combination of nutrients and enteric modulators several days prior to hatching, improves the growth performance of the chicks as much as by 5%-10% at marketing, 35-42 days after hatching. The composition of nutrients of the invention, include in one embodiment an amino acid, or a bioactive protein in another embodiment, or a bioactive polypeptide in another embodiment, or a bioactive peptide in another embodiment, or a bioactive hormone in another embodiment, or a carbohydrate in another embodiment, or a combination thereof in another embodiment. In one embodiment, the enteric modulator is hydroxymethylbutyrate.

Therefore according to this aspect of the invention and in another embodiment, supplementing such amniotic fluid of the pre-hatched egg with the optimal quantities of a combination of one or more bioactive proteins, or in one embodiment, with one or more nutrients or in another embodiment, with one or more enteric modulators, or in another embodiment, a combination thereof, enhances the growth performance of the hatched chick as encompassed within the scope of the methods and compositions of the invention as described herein. In one embodiment, the supplement is protected, or in another embodiment, unprotected.

In one embodiment, the term, “Protected” refers to the at least one health promoting, non-nutrient, bioactive protein being encapsulated in at least one layer, or in another embodiment, more than one layer, in a manner that, when a liquid/drink in one embodiment or feed in another embodiment, containing such bioactive protein is consumed by an avian infant, said encapsulation protects the bioactive protein, at least partially, during its passage through the two stomachs, such that sufficient amounts of the bioactive protein are still bioactive and are capable of driving the required positive health promotion or growth promotion benefits, as described herein.

In another embodiment, the term “Unprotected” or “unprotected” refers to conditions where no encapsulation and/or protection whatsoever is provided to the at least one health promoting, non-nutrient, bioactive protein, so that when a liquid/drink or feed containing such bioactive protein is consumed by an avian infant, said bioactive protein is degraded during its passage through the two stomachs, such that sufficiently high amounts of the bioactive protein must be supplemented in one embodiment, into the drink/feed, so sufficient quantity of the bioactive protein survives the passage through the two stomachs, and thus can drive the required positive benefits in another embodiment, as described herein.

In one embodiment, supplementing the amniotic fluid of the pre-hatched chick in-ovo facilitates enabling a newly hatched avian species' chick to reach improved weight gain within 5-9 weeks from hatching of at least 1.5% comparing with the industry standard, or in another embodiment, facilitating an improved feed conversion ratio of at least 1.5% comparing with the industry standard, or in another embodiment, facilitating greater daily, weekly or periodic feed intake which is at least 1.5% greater than the industry standard, or in another embodiment, facilitating the best feed conversion ratio possible, so the cost/performance ratio in growing Avian species achieved is at least 1.5% comparing with the industry standard, or in another embodiment, maximizing successful hatching percentage (e.g. the number of live healthy chicks hatched relative to the total number of eggs fertilized and incubated until hatching) of at least 1.5% comparing with the industry standard, or in another embodiment, minimizing the post hatching death rate (e.g. the number of adult Avian species' reaching marketing relative to the number of live chicks hatched) of at least 1.5% comparing with the industry standard, or in another embodiment, minimizing the epidemic disease episodes of Avian species during growing until reaching marketing, which reduces the cost of growing in at least 1.5% comparing with the industry standard, minimizing the systemic disease episodes of Avian species during growing until marketing, so percentage of Avian species disqualified from being marketed is minimal, in at least 1.5% comparing with the industry standard.

In another embodiment, supplementing avian species' nutrient intake is done with the composition of any appropriate embodiment described herein as described in the methods hereinbelow.

In one embodiment, the invention provides a method for an in-ovo administration of at least one health promoting, non-nutrient bioactive protein into the pre-hatching amniotic fluid.

In another embodiment, the invention provides a method for an In-ovo administration of a combination of at least two of the following: (a) at least one bioactive protein (b) at least one nutrient (c) at least one enteric modulator, into the pre-hatching amniotic fluid.

In one embodiment, the invention provides a method for an administration of at least one health promoting, non-nutrient bioactive protein into the drinking water of post-hatched avian chicks, or in another embodiment, to the special post-hatching feed immediately following hatching, until such avian chicks are 2-3 days old

In one embodiment, the invention provides a method for an administration of at least one health promoting, non-nutrient bioactive protein into the drinking water of post-hatched avian chicks starting 2-3 days after hatching until such chicks are 14-15 days old

In one embodiment, the invention provides a method for an administration of at least one health promoting, non-nutrient bioactive protein into the regular feed of post-hatched avian chicks starting on days 2-3 days after hatching until such chicks are 14-15 days old

In one embodiment, the invention provides a method for an administration of at least one health promoting, non-nutrient bioactive protein into the drinking water of post-hatched avian chicks starting 14-15 days after hatching until such chicks are mature and ready for marketing at 35-69 days old

In one embodiment, the invention provides a method for an Administration of at least one health promoting, non-nutrient bioactive protein into the regular feed of post-hatched avian chicks starting 14-15 days after hatching until such chicks are mature and ready for marketing at 35-69 days old.

In one embodiment of the invention, the newborn formulation may be an infant formula or a milk replacer/substitute or semi-solid feed or solid feed for mammal's newborn consumption.

In another embodiment, the term “milk replacer/substitute” refers to any milk replacer/substitute for mammalian neonates wherein the mammals are of the human, bovine, equine, and swine families for examples calf, lamb, pig, cows, sheep, goat, yaez, cats, dogs and horses. In one embodiment, the milk replacer/substitute refers to any milk replacer/substitute, suitable for mammalian neonates, wherein the mammals are of the feline and canine families.

In one embodiment of the invention, the semi-solid feed or solid feed is for any mammalian animal neonates, avian neonates or chordata neonates.

In another embodiment the plant-extracted bioactive compound is encapsulated in a matrix material, capable of being plasticized in one embodiment at low temperatures by a liquid plasticizer or in another embodiment, by liquid encapsulant component, which may be in another embodiment, a plasticizable biopolymer.

In one embodiment, the plasticized material is a carbohydrate polysaccharides, such as in another embodiment, pentosans, or in another embodiment, a physically or chemically modified starch or in another embodiment, cyclodextrin or in another embodiment mixtures thereof.

In another embodiment, the plasticized material is a polymer such as polyvinylpyrrolidone (PVP, Povidone) or other non-hydrophobic polymers such as N-vinylpyrrolidone (NVP) and poly(vinyl)acetate copolymers, (polyvinyl)alcohol chitosan or mixtures thereof. In one embodiment, the plasticized material is cellulose esters, cellulose ethers, and polyethylene glycol. In another embodiment, the plasticized material is a hydrocolloid such as xanthan, carragenan, alginate, gum arabic, gum acacia, gum tragacanth, gum conjac or in another embodiment, a mixtures thereof.

In one embodiment, the plasticized material is glutenins or in another embodiment gliadins, such as in one embodiment, vital wheat gluten or in another embodiment, isolated gluten, or in another embodiment zein, or in another embodiment vegetable or in another embodiment proteins such as protein from soy in one embodiment or milk in another embodiment, or in another embodiment mixtures thereof.

In another embodiment of the present invention, starches that used in the present invention are physically or in another embodiment chemically modified starches, with amylose/amylopectin ratios of between about 1 to about 0.001, derived from corn, or in another embodiment wheat, or in another embodiment rice, or in another embodiment potato, or in another embodiment tapioca, or in another embodiment yuka or in another embodiment arrow root or in another embodiment, a combination thereof.

In one embodiment, sources of starch which may be used also include flours from cereals such as corn, or in another embodiment, wheat, or in another embodiment durum wheat, or in another embodiment rice, or in another embodiment barley, or in another embodiment oat, or in another embodiment rye, or in another embodiment, mixtures thereof.

In another embodiment, only wall material approved by the FDA or similar regulatory body of the European Community and elsewhere shall be used.

In one embodiment compounds that can be used for forming the capsule walls are on the GRAS list.

In one embodiment of the present invention, any other food-grade or feed-grade encapsulating material, which has been approved by a recognized regulatory body for human and/or animal consumption (as applicable), shall serve as the encapsulation material in the process.

In one embodiment of the present invention, the wall material used is poly (DL-lactide-co-glycolide).

In another embodiment of the invention the food-grade or feed-grade encapsulating material, used in the neonate formulation comprises, polysaccharide, maltodextrin, milk powder, whey protein, lipid, gum, cellulosics or combinations thereof.

In one embodiment of the invention the plant-extracted bioactive compound being encapsulated or embedded maintains or in another embodiment, substantially maintains its biologically bioactive function and properties during the process of formulating the nutritional formulation, or in another embodiment during the normal shelf-life of the nutritional formulation in which it is incorporated.

In one embodiment of the invention the plant-extracted bioactive compound is analogous to glycoprotein, or in another embodiment to immunoglobulin, or in another embodiment to a protein, or in another embodiment to a peptide, or in another embodiment to a polypeptide, or in another embodiment to a hormone, or in another embodiment to an enzyme, or in another embodiment to a functional derivative thereof.

In another embodiment of the invention, the bioactive compound is analogous to insulin, or in another embodiment to IGF-I, or in another embodiment to IGF-2, or in another embodiment to EGF.

In one embodiment of the invention the bioactive compounds are analogous to alpha-1-proteinase inhibitor, or in another embodiment to alkaline phosphatase, or in another embodiment to angiogenin, or in another embodiment to antithrombin III, or in another embodiment to chitinase, or in another embodiment to extracellular superoxide dismutase, or in another embodiment to Factor VIII, or in another embodiment to Factor IX, or in another embodiment to Factor X, or in another embodiment to fibrinogen, or in another embodiment to glucocerebrosidase, or in another embodiment to glutamate decarboxylase, or in another embodiment to human serum albumin, or in another embodiment to myelin basic protein, or in another embodiment to lactoferrin, or in another embodiment to lactoglobulin, or in another embodiment to lysozyme, or in another embodiment to lactalbumin, or in another embodiment to proinsulin, or in another embodiment to soluble CD4, or in another embodiment to component and in one embodiment complexes of soluble CD4, or in another embodiment to tissue plasminogen activator and in one embodiment, a variant thereof or in another embodiment to combinations thereof and in one embodiment, a pharmaceutically acceptable salts thereof. The variants typically exhibit the same qualitative biological activity as the naturally occurring analogue, although variants can also be selected which have modified characteristics rendering them specifically adequate for use in specific mammals.

In one embodiment, the plant-extracted bioactive compound of the invention occurs naturally within a plant, or in another embodiment, a result of genetic modification, or in another embodiment, the result of genetic engineering.

In one embodiment of the invention the newborn formulation comprises uniformly sized particles of encapsulated plant-extracted bioactive compound, wherein the particles have a diameter between about 0.1 and about 5,000 micrometers. In one embodiment, D_{3,2} is the area average particle diameter.

D_{3,2} is a measure of average particle diameter and in another embodiment follows a log normal distribution. In one embodiment the term “D_{3,2}” refers to the average diameter of the particles calculated assuming spherical particles and inferring the average diameter from the surface area exposed to the measuring device. In one embodiment particle passing through a lit slit interrupt the light passage, wherein in another embodiment the interruption is tabulated and converted to diameter.

In one embodiment, log normal distribution has the following frequency distribution formula:

$$df = \frac{1}{σ \sqrt{2 π}} exp [ - \frac{{( {d}_{p} - \overline{{d}_{p}} )}^{2}}{2 {σ}^{2}} ] {dd}_{p}$$

### Wherein:

dₚ is the pore diameter in μM
d_{p bar} is the average pore diameter
σ is the standard deviation of pore sizes in μM

In one embodiment, particle size average, and in another embodiment, the standard deviation of the particle sizes is specifically designed for a given application. In one embodiment, the methods of producing the particles of the invention further comprise a step for increasing the particle size obtained in each step of the methods of processing in the invention.

In one embodiment, the term “Agglomerate” or “agglomeration” refers to a product (or a technique) that combines micron sized particles to form larger particles which are held together by a variety of physical-chemical forces. Agglomeration refers in another embodiment to the preparation of relatively larger particles by combining a number of relatively smaller particles into a single unit. Processes for accomplishing agglomeration are more fully discussed below. In one embodiment a high intensity agglomerator is used for the process of the present invention. In another embodiment, the terms spheroidal and substantially spherical are synonymous. In one embodiment, an agglomerating agent is used to affect the agglomeration. In another embodiment, the term “Agglomerating agent” refers to a composition used to effect agglomeration of fine powders, or in another embodiment, a dissolution agent is used wherein a blend of food grade emulsifiers that, when added to the binder solution used in the agglomeration process, results in an Nutritional formula, which readily dissolves when mixed with water or other suitable liquid. In another embodiment, the dissolution agent aids in dispersion and ultimate dissolution in water of the particles used to make the nutritional formula.

Many specialized processes and types of processing equipment have been developed for the agglomeration of particulate solids. (See generally, Pintaufo, N.D., COFFEE SOLUBILIZATION COMMERCIAL PROCESSES AND TECHNIQUES, Noyes Data Corporation, “Agglomeration Techniques”, pp. 177-209, (1975)). However, the same basic operating principles are involved in practically all cases. An agglomerating fluid, which is in one embodiment oil, or in another embodiment liquid water or steam, is uniformly distributed throughout the particles to be agglomerated, causing part or all of the particles to become tacky. The particles are then agitated, allowing the tacky particles to contact and adhere to other particles. Proper control of the amount of agglomerating fluid and the type and time of agitation will provide control over the final size of the agglomerated product.

In one embodiment of the present invention, solid feed formulation as used herein means a formulation able to maintain its density at room temperature and support its own weight.

In another embodiment of the invention, semi-solid formulation as used herein means formulations capable of flowing under their own weight, with viscosities between about 1 to about 600,000 Pascal seconds.

In one embodiment of the present invention, the formulation is being used for post weaning mammals.

In another embodiment, post-weaning mammals as used herein refers to the age at which the intensively grown mammals are typically weaned off the mother's milk. For example, intensively grown lambs are typically weaned between 25-35 days from birth. Intensively grown piglets are typically weaned between 18-30 days from birth; Intestively-grown calves are typically weaned between 40-70 days from birth.

In all of these newborn animals, in one embodiment of the invention, the provided quantity of the milk replacer containing the bioactive compound is gradually reduced, and the quantity of the bioactive compound in mix, pellets or other semi-solid or solid feed is gradually increased.

In another embodiment, the integration of the bioactive compound in mix/pellets/drink is advantageous for as long as 1-9 months post-birth or in one embodiment post-weaning. In another embodiment the bioactive compound is beneficial for 1-2 months, or in another embodiment, for 2-3 months, or in another embodiment, for 3-4 months, or in another embodiment, for 4-5 months, or in another embodiment, for 5-6 months, or in another embodiment, for 6-7 months, or in another embodiment, for 7-8 months, or in another embodiment, for 8-9 months post-birth or in one embodiment post-weaning.

In one embodiment of the invention, the solid or semi-solid feed formulation may be in the form a mash, or in another embodiment pellets, or in another embodiment granules, or in another embodiment agglomerate, or in another embodiment extrudate or in another embodiment combinations thereof.

In another embodiment of the invention, the bioactive compound being encapsulated or embedded maintains or substantially maintains its biological function during the digestion of the food or feed.

In one embodiment of the invention, the bioactive compound being encapsulated or embedded is released upon contact with a liquid.

In one embodiment of the invention, the solid or semi-solid feed formulation is a protein, or in another embodiment a glycoprotein, or in another embodiment an immunoglobulin, or in another embodiment a peptide, or in another embodiment a polypeptide, or in another embodiment a hormone or in another embodiment an enzyme, or in another embodiment a combination thereof.

In another embodiment of the invention, the newborn animal solid or semi-solid feed formulation comprises uniformly sized particles of an encapsulated bioactive compound, wherein the particles have an average size of between about 10 to about 4000 micrometers.

The formulations used in one embodiment of the invention are efficient for increasing the rate of weight gain or in another embodiment improving the FCR (Feed Conversion Ratio) of newborn animals, or in another embodiment reducing the mortality rate of newborn animals, or in another embodiment preventing diarrhea or in another embodiment gastric disorders or in another embodiment for increasing the life expectancy of newborn animals after birth.

Products containing protected bioactive compounds according to another embodiment are consumed by a variety of subjects such as in one embodiment, preterm infants, or in another embodiment post-discharge preterm infants, or in another embodiment term infants, or in another embodiment babies, or in another embodiment toddlers, or in another embodiment children, or in another embodiment adolescents, or in another embodiment adults, or in another embodiment elderly humans, or in another embodiment the infants or in one embodiment adults of non-human animals, such as in one embodiment bovine, or in another embodiment porcine, or in another embodiment caprine, or in another embodiment feline, or in another embodiment canine, or in another embodiment equine, or in another embodiment avian or in another embodiment aquaculture species or in another embodiment infants or adults of any other non-human animals.

In one embodiment of the invention, formulas and milk replacers for preterm infants, specially preterm infants born between weeks 24-36, where such formulas or milk replacers contain in one embodiment a protected or in another embodiment un-protected bioactive compound or in another embodiment, are supplemented with a protected or non-protected bioactive protein prior to consumption, are used to assist in accelerating the development in one embodiment or maturation of the preterm infant's gastrointestinal tract in another embodiment or in another embodiment, to prevent, or in another embodiment, to reduce the incidence of frequently fatal diseases associated with premature birth, such as NEC (Necrotizing Enterocolitis).

In another embodiment, foods and drinks of preterm or term infants incorporating a protected or in another embodiment, unprotected bioactive protein, when provided immediately in one embodiment, or shortly after birth in another embodiment, assist in eliminating or in another embodiment, reducing the onset of autoimmune diseases such as IDDM, or Celiac in another embodiment, or Inflamatory Bowel Disease in another embodiment, or Crohn's Disease in another embodiment, etc.

In one embodiment, a protected bioactive protein is premixed and packaged in a separate package from the food or feed or drink, or in another embodiment, prior to consumption by a subject, the package containing the protected bioactive protein is opened, and the protected bioactive protein is incorporated into the food or feed or drink of a subject, thus creating a bioactive supplemented food or feed or drink of a subject.

In another embodiment of the invention a method for encapsulating and embedding a bioactive compound in mammalian newborn formulation is provided, comprising the steps of, (i) mixing the bioactive compound with an edible food grade or feed grade or pharma grade encapsulating material forming a liquid blend; (ii) drying of the liquid blend; (iii) coating the dry blend with a additional food grade or feed grade or pharma grade encapsulating material layer; and (iv) adding the dry blend to the newborn formulation.

In one embodiment the mammalian newborn food formulation may be infant formula or milk replacer/substitute or other drink. Such a formulation is in another embodiment, a form of powder, a solution, a suspension, an emulsion, an ointment, a cream in both liquid, semi-solid or a solid form

In another embodiment of the invention, a formulation for post weaning mammals which is a solid or a semi-solid formulation is provided, comprising a encapsulated and embedded bioactive compound prepared by the following process: (i) mixing the compound with a food grade or feed grade or pharma grade encapsulating material so as to form a liquid blend; (ii) drying of the liquid blend so as to form a dry blend; (iii) coating the dry blend with a additional food grade or feed grade or pharma grade encapsulating material layer; and (iv) adding the dry blend to the mammalian solid or semi-solid feed formulation. The solid or semi-solid formulation may be in a form of pellets or mash/mix.

Further, according to one embodiment of the present invention, the step of mixing the bioactive compound and the wall forming food grade or feed grade or pharma grade material, involves the addition of liquid, such as, but not limited to: water, saline, alcohol, molasses, organic solvents or similar food grade or feed grade or pharma grade encapsulating material solvent.

In another embodiment of the present invention, the ratio between the food grade or feed grade or pharma grade material and the solvent of the food grade or feed grade or pharma grade encapsulating material may be in one embodiment of the invention between about 1:1 to about 1:1,000.

In one embodiment of the invention the ratio between the food grade or feed grade or pharma grade material and the solvent of the food grade or feed grade or pharma grade encapsulating material is between 1:3 and 1:100.

In another embodiment of the invention, the dry blend undergoes further size-reduction.

The encapsulated bioactive compound in one embodiment may be further encapsulated by an additional protection layer, which may be formed in another embodiment of the same food grade or feed grade or pharma grade encapsulating material or, in another embodiment a different food or feed grade or pharma grade encapsulating material. In one embodiment, the role of the protective layer is to protect the core from adverse environmental conditions such as temperature, or steam in another embodiment, or pressure in another embodiment, or other environmental triggers as described herein and their combination in another embodiment. In one embodiment, the protective layer's role is to protect the core from degradation properties to triggers. In one embodiment, each combination of a different number and type encapsulation layers result in a unique product suitable for the unique combination of the bioactive compound encapsulation manufacturing conditions, or in another embodiment the integration into food or feed or drink products, or in another embodiment, the the storage conditions, or in another embodiment the gastrointestinal system maturity, properties and characteristics of the subject at the specific age it is being fed. Accordingly and in one embodiment, a different multi-layer encapsulation is required for a piglet of 2 days old, comparing with the multi-layer encapsulation required for a 25 days old piglet. in another embodiment.

In one embodiment the dry blend is further mixed with said food or feed grade or pharma grade encapsulating material so as to form another layer of food grade or feed grade or pharma grade encapsulating material layer enveloping the bioactive compound.

In one embodiment of the invention the a bioactive compound may be identical or similar or analogous in its bioactive properties to alpha-1 proteinase inhibitor, alkaline phosphatase, angiogenin, antithrombin III, chitinase, extracellular superoxide dismutase, Factor VIII, Factor IX, Factor X, fibrinogen, glucocerebrosidase, glutamate decarboxylase, human serum albumin, myelin basic protein, lactoferrin, lactoglobulin, lysozyme, lactalbumin, proinsulin, soluble CD4, component and complexes of soluble CD4, tissue plasminogen activator or variant, pharmaceutically acceptable salt or combination thereof.

In another embodiment of the invention, the a food grade or feed grade or pharma grade encapsulating material is a polysaccharide, milk powder, whey protein, lipid, gum Arabic microcrystalline cellulose, their analogs or combinations thereof.

In one embodiment of the invention the a food grade or feed grade or pharma grade encapsulating material, is a solid at temperatures of up to 85° C.

In another embodiment of the invention, the step of drying the food grade or feed grade or pharma grade encapsulating material and a bioactive compound is done using the methods including but not limited to; freeze drying, vacuum drying, spray drying, osmotic dehydration, fluidized bed dehydration, solvent evaporation dehydration, sonication assisted dehydration, microwave-assisted dehydration, RF-assisted dehydration, either alone or commercially acceptable combinations thereof.

In one embodiment of the invention, the liquid mix is lyophilized after incorporating a bioactive compound and a food grade or feed grade or pharma grade encapsulating material ingredient.

In one embodiment lyophilization produces particles containing a protected bioactive compound and a food grade or in another embodiment feed grade or in another embodiment a pharma grade encapsulating material in a glassy matrix.

In one embodiment, a flash freezer is employed to dry the liquid mix through the utilization of liquid gas, which is, in one embodiment, nitrogen, or in another embodiment CO₂, or in another embodiment Propane, or in another embodiment, any suitable compressible refrigerant gas.

In one embodiment, the size of the droplets will vary between about 10 and about 5,000 micrometers.

In another embodiment the droplets size distribution depends on a variety of parameters such as in one embodiment, freeze sprayer nozzle size, or in another embodiment liquid gas temperature, or in another embodiment chamber temperature, or in another embodiment mix components ratio, or in another embodiment mix and gas flowrates, or in another embodiment encapsulating food grade or feed grade or pharma grade material concentration, or in another embodiment plasticizer type or in another embodiment freeze chamber wall geometry.

In one embodiment of the invention, the size distribution of the glassy droplets resulting from the process ranges between 50 microns and 1,000 microns.

In one embodiment this treatment results in glassy frozen micro droplets, where each micro droplet contains a protected bioactive compound, a food grade or feed grade or pharma grade encapsulating material and the food grade or feed grade or pharma grade solvent.

In another embodiment once such frozen droplets are placed in temperatures above the melting temperature of the mix, the liquid mix from the previous phase of the process shall be reconstituted.

In one embodiment of the invention, the process further includes the freeze-drying of a combination of a bioactive compound and a food grade or feed grade or pharma grade encapsulating material.

In another embodiment, freeze drying may be carried out on either a liquid mixture of a bioactive compound ingredient and a food grade or feed grade or pharma grade encapsulating material or on frozen glassy micro droplets as described hereinabove.

In one embodiment the result of this freeze drying process is dry glassy material which includes a food grade or feed grade or pharma grade encapsulating material and the a plant-extracted bioactive compound ingredient.

In another embodiment, freeze drying is performed on a liquid mixture, the result of the process was bulk dry material, porous by nature, containing a glassy matrix of the dried food-grade or feed grade or pharma grade encapsulating material encapsulating the plant-extracted bioactive compound.

In one embodiment, freeze-drying is performed on the output of the flash freeze spraying process, resulting in glassy droplets, with the food grade or feed grade or pharma grade encapsulating material incorporating the plant-extracted bioactive compound.

In another embodiment, low-temperature spray drying of combination of a bioactive compound and a food grade or feed grade or pharma grade encapsulating material is carried out.

In one embodiment, the bioactive compound was dispersed in the food grade or feed grade or pharma grade encapsulating material and atomized at a maximum temperature of 45° C.

In another embodiment, the maximum temperature is 37° C., preventing denaturation of the bioactive compound. In one embodiment, spray drying may be carried out on a liquid mixture of a protected bioactive compound, a food grade or feed grade or pharma grade encapsulating material and a chaperon-like protecting protein, resulting in dry material which comprises the food grade or feed grade or pharma grade encapsulating material and the a bioactive compound.

In one embodiment of the invention, the dehydration of the food grade or feed grade or pharma grade encapsulating material and the a bioactive compound conducted at a temperature, which is preferably below the denaturation temperature of any of the bioactive compound, when that bioactive compound is a protein, a peptide, a polypeptide or hormone.

In another embodiment, the dehydration of the food grade or feed grade or pharma grade encapsulating material and the bioactive compound is carried out at temperature below the onset temperature for the bioactive compound's denaturation threshold or degradation threshold.

In one embodiment of the invention, the dehydration process of the food grade or feed grade or pharma grade encapsulating material and the bioactive compound is carried out at a maximum temperature of 50° C.

In another embodiment of the invention, the step of drying the liquid blend results in glassy freeze-dried droplets containing a plant-extracted bioactive compound and a food grade or feed grade or pharma grade encapsulating material.

In one embodiment of the invention the step of freeze-drying is preceded by a step of spraying the liquid blend through an atomizer in the presence of a liquid gas.

In one embodiment, extrusion is used as an encapsulation method in which a core material is dispersed in a liquid mass of a bioactive compound and a food grade or feed grade or pharma grade encapsulating material and ultimately formed into microcapsule.

In another embodiment of the invention, encapsulating or embedding a protected bioactive compound in the formulation described above involves an additional step of premixing the blend in a small volume of the newborn formulation or food grade or feed grade or pharma grade encapsulating material, or semi solid or solid formulation, to ensure homogeneity prior to its mixing with the whole formulation.

In one embodiment of the invention, protection processes suited for use as used herein include, but are not limited to those which produce a protected bioactive compound in the form of a: powder, a micro-encapsulated powder, a nano-encapsulated powder, a liquid, a micro-emulsified liquid, a nano-emulsified liquid, a solution, a micro-emulsified solution, a nano-emulsified solution, a spread, a mash, an ointment, micro droplets, nano-droplets, tablets and solids such as for example, pellets.

In another embodiment of the invention, the encapsulation process includes duplex, W/O/W, O/W/O, double or multiple emulsions.

In one embodiment of the invention, the mix of a bioactive compound and a food grade or feed grade or pharma grade encapsulating material and a surfactant selected from the group of surfactants having an HLB value substantially below 7 are suspended in a non-miscible, food grade or feed grade or pharma grade material and further mixed affecting size reduction using methods hereinabove mentioned.

In another embodiment, the milled emulsion is further mixed with a food grade or feed grade or pharma grade material that is miscible with the food-grade or feed grade or pharma grade encapsulating material and a food grade or feed grade or pharma grade surfactant selected from the group of surfactants having an HLB value substantially higher than 7 and further reduced in size using one of the methods hereinabove mentioned.

According to an embodiment of the invention, following formulation of a bio active compound, micro emulsification or nano emulsification of the bioactive compound is conducted.

In one embodiment, the formulated bioactive compound is mixed with an emulsion incorporating water, oil phase and surfactant. As a result of such mixing, the bioactive compound's molecules are reorganized into the dispersed phase of the emulsion.

The protection provided to the bioactive compounds by the micro emulsion or nano emulsion in another embodiment, relates to temperature exposure protection, and improved solubility of the bioactive compounds within the food or feed with which it is integrated, following the release of the bioactive compounds from its encapsulation prior to its consumption and/or during the digestion process.

In another embodiment, the bioactive compound in the nano emulsion or micro emulsion is initially protected within the liquid micro emulsion or liquid nano emulsion.

A person holding ordinary skill in the art would readily recognize that this invention is not limited in its application to the details of construction and the arrangement of components set forth in the hereinbelow mentioned description. It should be appreciated that various modifications can be made without materially changing the scope or spirit of the current invention. It should be noted that practicing the invention not limited to the to the applications hereinbelow mentioned and many other applications and alterations may be made without departing from the intended scope of the present invention Also, it is to be understood that the lexicography employed herein is for the purpose of description and should not be taken as limiting.

In one embodiment of the invention, a method is provided for the encapsulation of a bioactive compound in a food grade or feed grade or pharma grade glassy matrix, the method comprising; (i) mixing a homogeneous intimate mixture between a bioactive compound and a wall forming, food grade or feed grade or pharma grade encapsulating material creating a blend, (ii) mixing said blend with an appropriate plasticizer, (iii) rapidly removing said plasticizer while inhibiting crystallization of the wall forming material thereby resulting in encapsulation of the bioactive compound in a food grade or feed grade or pharma grade glassy matrix.

In another embodiment of the invention, a method is provided for the encapsulation of a bioactive compound, comprising; (i) mixing a bioactive compound with a molten, a wall-forming food grade or feed grade or pharma grade encapsulating material, and (ii) rapidly cooling the molten, a wall forming material thereby resulting in encapsulation of the bioactive compound in a food-grade or feed-grade or pharma-grade glassy matrix.

In one embodiment “glassy-state matrix” refers to an amorphous metastable solid wherein rapid removal of a plasticizer causes increase in viscosity of the biopolymer to the point where translational mobility of the critical polymer segment length is arrested and alignment corresponding to the polymer's inherent adiabatic expansion coefficient is discontinued.

Hydrophilic materials, both of a monomeric and a polymeric nature either exist in one embodiment as or in another embodiment can be converted into amorphous states which exhibit the glass/rubber transitions characteristic of amorphous macromolecules. These materials have well defined glass transition temperatures Tg which depend in one embodiment on the molecular weight or in another embodiment on the molecular complexity of the glass forming substance. Tg is depressed by the addition of diluents. Water is the universal plasticiser for all such hydrophilic materials. Therefore, the glass/rubber transition temperature is adjustable by in one embodiment the addition of water or an aqueous solution, or in another embodiment, the removal of water or an aqueous solution.

In another embodiment, the plasticizer may be any substance of molecular weight lower than that of the biocompatible polymer that creates an increase in the free interstitial volume. In one embodiment, the plasticizer is an organic compound, which in one embodiment is triglyceride of varying chain length, or in another embodiment, the plasticizer is water.

In another embodiment of the invention, a method for encapsulating and embedding a bioactive compound in newborn formulation is provided, the method comprising; (i) mixing the bioactive compound with a food grade or feed grade or pharma grade encapsulating material so as to form a liquid blend, (ii) drying of the liquid blend so as to form a dry blend; (iii) coating the dry blend with an additional layer comprised of a food grade or feed grade or pharma grade encapsulating material, where each such layer has different properties relating to environmental conditions durability and degradation, and (iv) adding the dry blend to the newborn formulation thereby being a method for encapsulating and embedding a bioactive compound in newborn formulations.

In one embodiment of the invention, a newborn formulation is provided comprising a bioactive compound being encapsulated or embedded in a food grade or feed grade or pharma grade encapsulating material.

In another embodiment of the invention, a method for encapsulating or embedding a bioactive compound in newborn solid or semi solid feed formulation or newborn drink is provided, comprising the steps of; (i) mixing the bioactive compound with a food grade or feed grade or pharma grade encapsulating material so as to form a liquid blend, (ii) drying of the liquid blend so as to form a dry blend, (iii) coating the dry blend with a additional layer comprised of a food grade or feed grade or pharma grade encapsulating material, where each such layer has different properties relating to environmental conditions durability and degradation, and (iv) adding the dry blend to the newborn animal solid or semi solid feed formulation of newborn drink thereby being a method for encapsulating and embedding the bioactive compound in newborn animal solid or semi solid feed formulation or newborn drink.

In another embodiment of the invention, a newborn animal solid or semi-solid feed formulation or newborn drink is provided, comprising a bioactive compound being encapsulated or embedded in a food grade or feed grade or pharma grade material.

The following examples are presented in order to more fully illustrate some embodiment of the invention. They should, in no way be construed, however, as limiting the scope of the invention.

### EXAMPLES

### Example 1

### Effects of Dietary Insulin Derivatives on Weight Gain and Feed Conversion Ratio in Poultry

### Materials and Methods

2,500 male Ross chicks, eight hours old were used in a study where different types of molecules derived from insulin were added during periods ranging between 7 and 35 days from hatching. The daily amount of bioactive material per chick ranged between nanograms/gram of feed to tens of nanograms of bioactive material/gram of feed. The bioactive insulin and insulin-analog material was provided via semi-solid feed and drinking water. The bioactive material, amino acids complex degraded ex-vivo from insulin, was of human, bovine, porcine and plant-extracted source and was protected by freeze drying or by fluid bed manufacturing techniques in a polysaccharide matrix before incorporation into the feed or drinking water.

### Results

Between 14 days and 35 days from hatching, the insulin and insulin degraded amino acids complex bio active compounds provided through the drinking water or feed resulted in a higher weight gain of the study groups by up to 6.1% compared to positive control, up to 3.5% in Feed Conversion Ratio, and increase of breast muscle weight in up to 3.0% of the study groups compared to a positive control.

### Example 2

### Extraction, Purification and Identification of Insulin-Like Bioactive Material from Momordica Charantia (Bitter Melon)

### Materials and Methods

2,500 grams of freeze dried Momordica Charantia fruit material was extracted, resulting in 20% of solids (i.e. 500 Grams of solids) comprised of salts and Momordica Charantia proteins. The crude extract was freeze dried, and then solubilzed and passed through filtering membrane for the removal of salts. The remaining extract was freeze dried, resulting in 75 grams of crude extract substantially salt-free (representing 15% solids from the original freeze dried Momordica Charantia fruit material). The final extract was tested using four methods and kits: HPLC analysis, LC-MS analysis, a bovine insulin ELISA kit, and a lymphoma cell line, which proliferates exclusively in the presence of insulin and insulin-like bioactive compounds.

### Results

### Compound Quantity:

Using HPLC analysis, the concentration of the bioactive insulin-like compound was found to be between 10⁻⁵ 10⁻⁸ of the weight of the fresh Momordica Charantia fruit. The retention time of the plant-extracted molecule was almost identical to the retention times of recombinant human insulin, bovine insulin and porcine insulin.

### Compound Identification:

LC-MS analysis found that the plant-extracted insulin-like compound is most similar to bovine insulin.

### Compound Quantification:

Bovine insulin ELISA kit analysis found the concentration corresponding to the measured quantitative activity of the plant-extracted insulin-like compound to be between 1:4 to 1:6 of the concentration as found by HP-LC analysis.

### Compound Bioactivity:

The plant-extracted insulin-like compound analyzed with Lymphoma cell line, and was found to have bioactivity which ranged from 1:2 to 1:10 of the corresponding values projected by the HP-LC analysis

### Example 3

### Fluidized Bed Coating Process Specifications

### Description of Coating Samples:

PMDI−Polycose (core)+[MD+Insulin](coating solution)−→(one concentration−2 IU/gr)

LMDI−Lactose (core)+[MD+Insulin](coating solution)−→(one concentration−2 IU/gr

MMDI−Maltodextrin (core)+[MD+Insulin](coating solution)−→(one concentration−2 IU/gr

MD 18 Concentrations of 10%, 20%, 30%

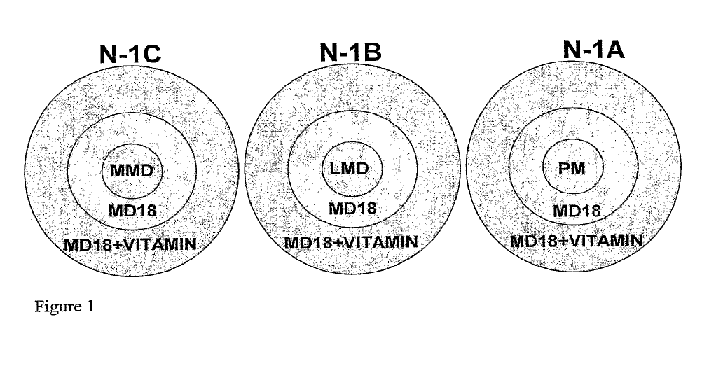

MD 18+Vitamin C 10% (one concentration on each core and MD18 coating) as shown in FIG. 1

### Coating Conditions

The mixing was done under food grade regulations conditions and with compliance with the Biodar ISO9001:2000 quality system procedures. During all manufacturing process, product temperature did not exceed 37° C. The process was performed at slow rate to prevent agglomerates.

### Sampling

From each stage in the process a sample of 10 grams is taken, packed in a bag and labeled to indicate the sample number.

### Example 4

### Insulin Solution Premix Preparation

### Mixing Conditions

Mixing was done under cGMP conditions and with compliance with HACCP procedures

### Solution Preparation

The Maltodextrin DE-18, insulin and Saline 0.45% solution was prepared using 20% Maltodextrin DE-18, Insulin (100 IU/ml) at a ratio of 10 cc to 500 gr active ingredient coated core (MD/Polycose core coated with MD+insulin layer). Saline 0.45% to complete 100% solution. Saline was added partially to the solution. The rest of the Saline solution was used to rinse the insulin bottles to ensure all material have been washed out and added to the solution.

The solution was mixed until the Maltodextrin was completely dissolved. FIG. 1 shows the multilayered encapsulation process used.

### Example 5

### InsuMeal™ Product

### In Vitro Testing

Several in-vitro tests were performed on the InsuMeal™ product in order to verify that the manufacturing process does not adversely affect the required product characteristics and bioactivity, and further to ensure that it consistently meets its technical specifications.

### Osmolarity Testing

The RTF (Ready-To-Feed) liquid formula has a defined osmolarity that is important for suitable nutrients consumption. Therefore, a test was performed to verify that the addition of InsuMeal™1.0 Grams of powder to the RTF does not change the osmolarity of the liquid formula.

The test was performed by immersing 1.0 g and 1.5 g of InsuMeal™ in 60 ml preterm RTF formula bottle, analyzing the osmolarity and comparing it to a control containing the same RTF formula without insulin. Each sample was analyzed in triplicates. The result are shown in table 1

TABLE 1
Final formula osmolarity

| | RTF Osmolarity (mOs) |
|---|---|
| Sample | Average |
| Control (RTF only) | 297.333 |
| 1.0 gr. (MD + Insulin) | 298.666 |
| 1.5 gr. (MD + insulin) | 303.5 |

The results indicate that the addition of InsuMeal™ powder to the RTF formula had no effect on the final solution osmolarity, remaining within its specifications.

### Insulin Performance

Insulin undergoes several processes during production. Several tests were performed to validate that its bioactivity is maintained following the process, as well as activated as soon as the product is immersed within a liquid solution. Furthermore, the insulin stability was tested after micro encapsulation following exposure of the product to high temperatures (up to 95° C.) and over time (up to 24 hr), to ensure product performance at extreme conditions.

Insulin performance was evaluated by both quantitative and qualitative methods:
i. Quantitative test performed by an immunology assay using human insulin Elisa kit [LINCO RESEARCH Cat. #EZHI-14K).
ii. Qualitative test: Done using an intended LB cells kit (21). LB cells which proliferates exclusively in the presence of insulin and insulin-like bioactive compounds. This growth is correlated to the bioactive insulin concentration and can be measured by spectrophotometric methods with a designated commercial kit.
   Insulin stability during InsuMeal™ technological process

Several experiments were carried out, in which InsuMeal™ product was manufactured with a pre-defined insulin concentration (100 μU). The InsuMeal™ powder was suspended in a solution and the insulin concentration was determined by using the Elisa kit and compared to the stoichiometric concentration of insulin. The average detected insulin concentration was found to be identical to the predicted/pre-prepared one with deviations of ±2.3% (average of 99.36 μU).

These findings indicate that the insulin is not damaged during the InsuMeal™ production process. The insulin is protected within its matrix and once solubilized, is completely released to the liquid medium.

### Product Stability in Liquid Infant Formula Over Time

The InsuMeal™ is intended to be consumed immediately after solubilization in the infant formula. Nevertheless, the insulin's stability over time was measured by adding a pre-defined quantity of liquid insulin (concentration of 100 μU per aliquot) to 60 ml Materna preterm infants formula bottle, and the insulin quantity was analyzed immediately following addition, then after 3, 6, 9, 12, 15, 18, 21 and 24 hours by using the Elisa kit. The results are shown in table 2. Furthermore, in order to evaluate the final product homogeneity, at each time interval, sampling was taken at the upper, middle and lower layers of the sampled RTF bottle, as well as after formula liquid stirring.

TABLE 2
Insulin 24 H stability overtime in RTF formula

| Time (hour) | Insulin concentration |
|---|---|
| from insulin addition | (μU) |
| 0 | 95.43 |
| 3 | 105.47 |
| 6 | 111.59 |
| 9 | 93.34 |
| 12 | 99.55 |
| 15 | 105.38 |
| 18 | 96.74 |
| 21 | 93.78 |
| 24 | 104.5 |

Results show that the insulin is highly stable for at least 24 hr from its addition to liquid infant formula. Insulin concentration different layers of the formula bottle showed the insulin is well distributed within the formula bulk and does neither cream or precipitate.

InsuMeal™ product with a pre defined insulin quantity (concentration of 100 μU) was added to lamb milk formula at 37-40° C. Insulin measurements after periods of up to 2 weeks showed the expected concentration ±2.5%, showing that the product effectively protects the insulin component and that the insulin is fully released once the product is added to liquid and is very stable over practical time.

### InsuMeal™ Product Stability at Extreme Temperatures

Since insulin is a temperature sensitive protein, the product ability to protect the insulin component following exposure of the encapsulated powder to high temperature for various durations, was measured.

InsuMeal™ powder with pre-defined insulin quantity (concentration of 100 μU) was directly exposed to several temperatures between 50-95° C. for various periods of up to 180 minutes. Each sample was then solubilized and tested for insulin concentration both quantitative (Elisa kit) and qualitative (LB cells) analytical kits.

Results showed that InsuMeal™ maintained its insulin component bioactivity after exposure to temperature of up to 90° C. for 7.5 minutes. These results indicate that InsuMeal™ micro-encapsulation process effectively protects the sensitive insulin.

### Influence on Vitamin C in the Preterm RTF Formula

Tests were done to ensure that the insulin does not affect available vitamin C concentration in the formula, since both are anti oxidants and insulin presence may negatively affect the vitamin concentration. The vitamin concentration was sampled at 0, 6 and 12 hours from initial insulin supplementation. No significant differences in vitamin C concentration were found.

### Example 6

### NovoMax™ (Animal Feed Supplement) Product

### In Vivo Testing

Studies using micro encapsulated NovoMax™ versions (insulin and amino acids complex derived from insulin) as feed additives in-vivo, in controlled environment and in a commercial field were carried out, encompassing over 16,000 chicks, piglets, calves and lambs. In these studies, newborn animals were given the bioactive feed additives additives to their drink/feed for different periods and in different dosing regimens.

Toxicity was evaluated by mortality rates, blood glucose and insulin levels (evaluating for any hypoglycemia or high insulin levels) and any active intake of the insulin (insulin residues in different body tissues). Efficacy was evaluated by calculations of Feed Conversion Ratio (FCR), which reflects the ratio between the amount of food consumed per animal vs. the final animal weight gain (FCR=amount of food consumed/animal weight), reflecting the animal's gastrointestinal system absorption efficiency and development.

### Poultry Study

2,016 1-day old chicks were divided into 6 test groups (amino acids complex degraded from insulin addition to feed) and a control group (no bioactive compound addition to feed). The chicks were fed with NovoMax™ for 21 days, and measurements were taken up to 37 days (marketing date). Glucose and insulin blood levels, as well as insulin concentrations in liver and muscle tissues were measured. Table 3 shows the glucose blood level and insulin serum and tissue concentration measurements.

TABLE 3
Blood and tissue tests of poultry experiments

| | Test | | | |
|---|---|---|---|---|
| | | Serum | | |
| | Glucose | insulin | Liver tissue | Muscle tissue |
| Group | (mg/dl) | (μU/ml) | insulin (μU/ml) | insulin (μU/ml) |
| Test group | 235.66 ± 7.4 | 7.90 ± 6.34 | 2.74 ± 0.63 | 1.63 ± 0.49 |
| (avg.) | | | | |
| Control | 241.75 ± 9.2 | 6.16 ± 7.5 | 2.72 | 1.99 |
| (avg.) | | | | |

Average blood glucose and insulin levels in the test and control groups were similar. The insulin concentrations in liver and muscle tissues in the test groups were also similar to those of the control group. Furthermore, the average mortality rates in the test groups were 30% lower than those in control. These findings show that addition of amino acids complex degraded ex-vivo from insulin to the poultry diet is safe and does not adversely affect glucose and insulin levels by hypoglycemia or hyperglycemia as well as no evidence of excessive intake of insulin by the body tissues. The reduced mortality rate supports the safety as well as indicating product effectiveness in promoting health effect associated with the amino acids complex intake.

Two additional NovoMax™ poultry studies which included 1,100 chicks were divided into a test group (insulin given in drinking water) and a control group (without insulin) were performed. The treatment and the follow up durations were identical to the above mentioned trial. Glucose and insulin blood levels are shown in table 4.

TABLE 4
Blood and tissue tests of poultry experiments

| | | Glucose |
|---|---|---|
| Trial name | Group | (mg/dl) |
| Bitzaron, Israel | NovoMax ™ treatment | 254.72 ± 15.26 |
| (Dec.) | Control | 240.4 ± 11.9 |
| Bizaron, Israel | NovoMax ™ treatment | 223.1 ± 30 |
| (Oct.) | Control group | 235 ± 26.07 |

Here also the data shows that NovoMax™ addition to the poultry's diet is safe and does not adversely affect the glucose and insulin levels by hypoglycemia or hyperglycemia.

### Swine Study

180 1-day old piglets were divided into 3 groups—the first group were fed NovoMax™ (amino acids complex degraded ex-vivo from insulin) as a feed additive to the drinking water, the second group were fed NovoMax™ (amino acids complex degraded ex-vivo from insulin) as a supplement to pre-starter pelleted feed, and a control group without any addition of any bio active compound (other than the naturally occurring in sow's milk). The piglets were fed for 25 days, which is a common period for piglet weaning, and were followed until marketing (168 days). During the 25 days treatment period, the piglets received NovoMax™ in bioactive compounds concentration equivalent to up to 5 times higher than the natural insulin concentration in the sow's colostrum. Before marketing, body liver and muscle tissues of several pigs were analyzed for insulin levels. Results are shown in table 5.

TABLE 5
tissue tests of swine experiments

| | Liver tissue | Jejunum tissue insulin |
|---|---|---|
| | insulin (μU/ml) | (μU/ml) |
| Study | 2.660 ± 0.98 | 1.874 ± 0.411 |
| group (water) | | |
| Study group (feed) | 3.14 ± 1.36 | 1.437 ± 0.837 |
| Control group | 2.44 ± 1.11 | 2.23 ± 1.104 |

The data shows no amino acids complex or insulin residue (beyond natural levels) was found neither in the jejunum or liver. Additional observation of this study showed the survival of seven (7) low weight newborn piglets (IUGR or Intra Uterine Growth Retarded) that regularly consist about 10-15% from the newborn piglet population, and do not typically survive. This result show the potential health effects of NovoMax™ (amino acids complex degraded ex-vivo from insulin) beyond growth and weight gain characteristics.

### Calves Study

48, 7-day old calves, post colostrum suckling stage, were divided into two groups: a test group receiving 600 μU/ml insulin which is within the normal values of insulin in bovine colostrum and a control group without insulin addition. The calves were treated for 40 days, and at the end, glucose and insulin blood levels were measured as well as blood count. Results are depicted in table 6. Hematological pictures of both the study and control group were similar.

TABLE 6
Blood and insulin tests of calves experiment

| | Glucose |
|---|---|
| | (mg/dl) |
| Insulin additive | 92.83 ± 16.14 |
| Control | 79.75 ± 17.56 |

As shown in table 6, the blood glucose levels in both the test and control groups were similar. These findings are supported since the provided insulin concentration is within the normal values of insulin in bovine colostrum, proving that the insulin addition is a safe supplement at the concentrations given.

### Lamb Studies

Three lamb studies were carried out, in which several concentrations of insulin were added to the test group milk replacer for 28 days from birth and compared to control groups fed with milk replacer with no insulin addition. On day 28 blood glucose and insulin levels were measured. The results are indicated in table 7.

TABLE 7
Blood and insulin tests of lambs experiments

| | | day 28 test | | | |
|---|---|---|---|---|---|
| | | | Serum | day 42 test | |
| Trial | | Glucose | insulin | Glucose | Serum insulin |
| name | Product | (mg/dl) | (μU/ml) | (mg/dl) | (μU/ml) |
| Gazit | NovoMax ™ 600 | 82.7 ± 8.07 | 876.8 ± 221.9 | 63.3 ± 3.6 | 101 ± 99.41 |
| | μU/ml | | | | |
| | Control group | 94.4 ± 15.02 | 971.2 ± 344.3 | 58.9 ± 3.2 | 185.44 ± 79.84 |
| Ilania | NovoMax ™ 200 | 107.5 ± 14.02 | | | |
| | μU/ml | | | | |
| | NovoMax ™ 400 | 103.3 ± 14.33 | | | |
| | μU/ml | | | | |
| | Control group | 111.6 ± 15.97 | | | |
| Zaid | NovoMax ™ 300 | 110 ± 10.39 | | | |
| | μU/ml | | | | |
| | Control group | 118 ± 9.29 | | | |

As shown in table 7, insulin addition to lambs' diet had no negative effect on their insulin and glucose blood levels compared to the control group, proving that the addition of insulin to lambs' diet at the tested concentrations is safe. The fact that the added insulin concentration was several folds higher than lambs full milk insulin concentrations adds to the supports of the product's safety.

### Human Studies

Clinical trials were done involving supplementation of preterm infant orally-fed with Insulin. Each preterm infant with 4 Units (e.g. 4,000,000 Micro-Units) of Insulin per Kg. per day for 28 days following delivery (e.g. up to 116,000 fold the natural quantity of Insulin provided through natural mother milk in the first few days from birth). The results of this trial showed preterm infants fed with Insulin at the above quantities, achieved full enteral feeding within 11 days—compared with 20 days in the control group; Lactase activity in the trial group was 13.3—compared with 6.5 in the control group; and; Gastric residuals were 22—compared with 54 in the control group. In summary, the preterm infant feeding supplemented by Insulin demonstrated significant health advantages to the preterm infants treated with therapeutic levels of oral Insulin.

### Example 7

### Cobb Broiler Chicks Fed with Various Dosing Regimens of Pelleted NovoMax™ Compared to the High Standard Commercial Pelleted Broiler Formulation

The objective of the experiment was to evaluate the effect of various regimens of pelleted NovoMax™ supplemented as a feed additive into a proven, high-performance AGP-enriched feed version, on the growth performance of Cobb broiler chicks.

### Materials

### Test Conditions

The test conditions were divided as seven doses of NovoMax™ premix (incorporating amino acids complex degraded ex-vivo from insulin) for pelleted broiler feed, in combination with seven different withdrawal dates, added to the commercial broiler diet, and one (positive) control group fed the normal high-standard broiler diet.

### Target Species

One-day-old, Cobb male chicks were the target species for this trial.

### Animals and Maintenance Conditions

Prior to the beginning of the experiment, the chicks were examined for any signs of ill-health and/or injury. Any bird appearing to be in poor health was removed from the experiment. The birds were assigned to their treatment groups on day 0. Each pen housing the birds was uniquely labeled.

### Environment

The birds were kept in 32 floor-pens (2.35 m×2.00 m), with wood shavings as bedding. The study facility was kept under the following environmental conditions:

| | |
|---|---|
| Temperature | Start 32° C. |
| | Finish 22° C. |
| Light | Week 1: 23 h/day |
| | Week 2: 14 h/day |
| | Week 3: 12 h/day |
| | Week 4: 6-10 h/day |

### Water Supply

Water was available ad libitum from bell drinkers (one per pen) throughout the study period.

### Experimental Design

### Assignment of the Treatments

The seven treatments and the control were allocated at equal weighting over the 32 pens (8 treatments×4 pen replicates) using a standard randomization technique (Table 1). Each pen contained 63 birds at the beginning of the study.

### Randomization Technique

To make sure that every pen will contain the same population dispersion, each bird was individually weighed before allocation and placed in groups of 2 gram weight segments (e.g. 38-39 g, 40-41 g, 42-43 g etc.). Groups of 63 chicks were formed, placing in each pen an identical number of chicks from each weight group. Using this method of distributing chicks into pens, any potential difference between pens, resulting from genetic differences, hatching time gaps, placement in the incubator and differences between incubator cells was eliminated.

### Administration of the Test Articles and Nature of the Treatments

The NovoMaxl™ premix was mixed with the best performing commercial broiler formula available from the feed mill (Miloubar, Haifa Bay, Israel). The different rations were delivered into each pen one day before the beginning of the trial.

The rations were administered continuously from day 0 through day 21 (day of InsuMeal™ withdrawal) and from day 22 to day 37 all 8 treatments received the control group feed formula.

The basal pre-starter diets were all based on corn and soybean meal to which: 3.6 IU, 2.376 IU and 1.188 IU equivqlents to porcine insulin (prior to ex-vivo degradation) based NovoMax™ was added.

The 8 treatments were as follows:

| | |
|---|---|
| 1. | Treatment #1: Control diet feed with the addition of coated |
| | NovoMax ™ premix - 3.6 IU (porcine insulin equivalent) |
| | of NovoMax ™ per chick. |
| 2. | Treatment #2: Control diet feed with the addition of coated |
| | NovoMax ™ premix - 3.6 IU (porcine insulin equivalent) of |
| | NovoMax ™ per chick. |
| 3. | Treatment #3: Control diet feed with the addition of coated |
| | NovoMax ™ premix - 2.376 IU (porcine insulin equivalent) of |
| | NovoMax ™ per chick. |
| 4. | Treatment #4: Control diet feed with the addition of coated |
| | NovoMax ™ premix - 1.188 IU (porcine insulin equivalent) of |
| | NovoMax ™ per chick. |
| 5. | Treatment #5: Control diet feed with the addition of coated |
| | NovoMax ™ premix - 3.6 IU (porcine insulin equivalent) of |
| | NovoMax ™ per chick. |
| 6. | Treatment #6: Control diet feed with the addition of coated |
| | NovoMax ™ premix - 2.376 IU (porcine insulin equivalent) of |
| | NovoMax ™ per chick. |
| 7. | Treatment #7: Control diet feed with the addition of coated |
| | NovoMax ™ premix - 1.188 IU (porcine insulin equivalent) of |
| | NovoMax ™ per chick. |
| 8. | Treatment #8: Basal treatment in which birds were fed on the basic |
| | pre-starter diet for the whole period, 1-37 days. Control group. |

Nature of encapsulation and mixing remix):

| | |
|---|---|
| 1. | Coating number 1, 540 g of coated NovoMax ™ premix with 3,957 g |
| | of corn powder, (preparation of 1,500 kg mash) mash number 1011. |
| 2. | Coating number 2, 783.3 g of coated NovoMax ™ premix with |
| | 3,712.69 g of corn powder, (preparation of 1,500 kg mash) mash |
| | number 1012. |
| 3. | Coating number 2, 519.62 g of coated NovoMax ™ premix with |
| | 3,980.38 g of corn powder, (preparation of 1,500 kg mash) mash |
| | number 1013. |
| 4. | Coating number 2, 259.81 g of coated NovoMax ™ premix with |
| | 4,240.19 g of corn powder, (preparation of 1,500 kg mash) mash |
| | number 1014. |
| 5. | Coating number 3, 787.3 g of coated NovoMax ™ premix with |
| | 3,712.69 g of corn powder, (preparation of 1,500 kg mash) mash |
| | number 1015. |
| 6. | Coating number 3, 519.62 g of coated NovoMax ™ premix with |
| | 3,980.38 g of corn powder, (preparation of 1,500 kg mash) mash |
| | number 1016. |
| 7. | Coating number 3, 259.81 g of coated NovoMax ™ premix with |
| | 4,240.19 g of corn powder, (preparation of 1,500 kg mash) mash |
| | number 1017. |
| 8. | Mash number 1010. |

Methods

Birds and their Allocation

A total of 2,016 one day-old Cobb male broiler chicks were allocated to 32 identical floor-pens (area 4.7 m²) such that there were 63 chicks in each of the pens. All birds were fed ad libitum on the 8 experimental diets from 0 to 21 days of age and on the proven commercial high performance formulation from 22 to 37 day of age. Treatment numbers, different encapsulations and concentrations of added NovoMax™ to the different diets are all shown in Table 11. Throughout the experiment the broilers were reared at stocking densities that were as similar as possible to those practiced commercially (13.40 birds/m²). All birds had free access to water and feed at all times.

### Diet Mixing and Sampling

All diets were mixed using a mixer. The diets did not contain any growth promoter or antibiotics other than those prescribed by the feed mill. The premixes contained encapsulated NovoMax™ and cornflower, were mixed with the pre-starter food and pelleted at Miloubar (processing plant).

The peak temperatures at which the diets were pelleted were 90° C.

Samples of each diet were collected manually after mixing and after pelleting. In addition, at day 21, samples of each diet were collected from the hoppers in the floor-pens for subsequent analysis.

### Analyses of the Test Articles in the Diets

Diets were analyzed for bioactivity content in the various concentrations, to confirm that the pelleting process didn't alter NovoMax™ bioactive compound.

### Blood Sampling

From each treatment group, three birds in the average weight of each pen were selected. From each bird, a blood sample was taken for direct glycemia test using a commercial glucometer (Roche Diagnostic) and for serum insulin.

A Total of 48 samples were taken: 24 samples on day 14 and 24 samples on day 21 from the start of the trial.

### Histology

From selected treatment groups, one chick from each group, in the average weight of the pen, was sacrificed.

Organ Sampling from Each Sacrificed Bird was Taken:
i. Jejunum after washing from food contents with saline and preservation in buffer formaldehyde 4%.
ii. Liver preserved in buffer formaldehyde 4%.
iii. Muscle sample of the breast, quadriceps femoral and internal muscle tibia, frozen in −20° C. until subsequent analysis.

### Biological Residue

From the treatment group and control presence of NovoMax™ bioactive ingredient was checked in the tissue from liver and muscle breast, the test was done by ELISA, at day 21 (Table 11).

TABLE 11
ELISA test, from the liver at 21 days

| | Concentration of | St. dev |
|---|---|---|
| Group | insulin (μU/ml) | (Approx.) |
| NovoMax ™ treatments | 2.74 | 0.63 |
| (Liver) | | |
| Control | 2.72 | |

### Observations Recorded During the Trial

### Feed Intakes

The feed intakes for each pen of birds were determined by weighing the feed in the hoppers on days 0, 14, 21, 28 and 35 (table 8).

TABLE 8
Feed intake (Kg/pen) between 0-14, 14-21, 21-28, 28-35 days
Age (DAYS)/Treatment

| | 1 | 2 | 3 | 4 | 5 | 6 | 7 | 8 |
|---|---|---|---|---|---|---|---|---|
| 0-14 | 34.500 | 35.000 | 33.750 | 33.750 | 34.250 | 32.000 | 33.250 | 32.500 |
| 14-21 | 39.745 | 37.915 | 40.635 | 38.660 | 40.475 | 39.950 | 36.910 | 38.610 |
| 21-28 | 62.639 | 60.814 | 61.088 | 62.380 | 61.559 | 61.601 | 62.237 | 59.422 |
| 28-35 | 69.706 | 67.650 | 68.351 | 72.004 | 71.360 | 68.424 | 73.065 | 65.610 |

Values are the means of 4 replicates per treatment

### Body Weight Gains and Feed Conversion Ratios

The body weight of the birds in each pen was also recorded on days 0, 14, 21, 28 and 35, immediately after each measurement of feed intake had been made. The body weight gains for the periods 0-14, 15-21, 22-28 and 29-35 days are calculated in (Table 9). From these and the corresponding feed intakes the feed conversion ratios of the birds on each of the 8 treatments and for each stage of growth (i.e. 0-14 days, 15-21 days, 22-28 days and 29-35 days) and overall (0-35 days) were calculated.

TABLE 9
Average Chicks Weight (g) at days 14, 21, 28 and 35

| Treatment | Day 14 | Day 21 | Day 28 | Day 35 |
|---|---|---|---|---|
| 1 | 488 | 961 | 1529 | 2217 |
| 2 | 482 | 942 | 1506 | 2181 |
| 3 | 480 | 953 | 1532 | 2226 |
| 4 | 464 | 919 | 1502 | 2211 |
| 5 | 470 | 915 | 1481 | 2126 |
| 6 | 462 | 913 | 1507 | 2172 |
| 7 | 459 | 919 | 1546 | 2182 |
| Control | 463 | 908 | 1481 | 2170 |

### Health and Conditions

The birds were examined daily in their pens and any variation in appearance and/or behavior was recorded. If a bird was in poor condition it was observed more frequently. If a bird was judged unlikely to survive or to be suffering pain or distress, it was liberated and date of death recorded.

### DISCUSSION

### Weight Gain

As shown in tables 8 and 10, between 0-14 days, birds from treatments 1, 2, 3 performed significantly better compared to the control (488, 482, 480, 463 g respectively). The remaining treatments did not differ from the control.

| | | Day 14 | Day 21 | Day 28 | Day 35 |
|---|---|---|---|---|---|
| Thermal 3.6 | Weight FCR | 5.40% | 5.84% | 3.24% | 2.17% |
| | | −1.30% | 2.93% | 0.53% | −0.30% |
| Thermal + | Weight FCR | 4.10% | 3.74% | 1.69% | 0.51% |
| Enteric 1-3.6 | | −3.30% | 3.01% | 0.68% | 3.17% |
| Thermal + | Weight FCR | 3.67% | 4.96% | 3.44% | 2.58% |
| Enteric 1-2.4 | | 1.30% | 2.93% | 0.46% | 2.09% |
| Thermal + | Weight FCR | 0.22% | 1.21% | 1.42% | 1.09% |
| Enteric 1-1.2 | | −2.60% | 2.71% | 0.99% | 3.35% |
| Thermal + | Weight FCR | 1.51% | 0.77% | 0.00% | −2.03% |
| Enteric 2-3.6 | | −4.08% | −2.71% | −2.18% | −2.33% |
| Thermal + | Weight FCR | −0.22% | 0.55% | 1.76% | 0.09% |
| Enteric 2-2.4 | | 1.13% | 0.15% | 0.79% | 0.18% |
| Thermal + | Weight FCR | −0.86% | 1.21% | 4.39% | 0.55% |
| Enteric 2-1.2 | | −5.12% | 2.56% | 2.38% | 0.78% |
| Control | Weight FCR | | | | |

BETWEEN 14-21 DAYS, BIRDS FROM TREATMENTS 1, 2, 3 PERFORMED SIGNIFICANTLY BETTER COMPARED TO THE CONTROL (961, 942, 953, 908 G RESPECTIVELY). THE REMAINING TREATMENTS COMPARED TO THE CONTROL ALSO PERFORMED BETTER (919, 915, 913, 908 G RESPECTIVELY) BUT LESS SIGNIFICANTLY COMPARED TO TREATMENTS 1, 2, 3

Between 21-28 days, birds from treatments 1, 2, 3, 4, 6, 7 performed better compared to the control (1529, 1506, 1532, 1502, 1507, 1546, 1481 g respectively). Treatment 5 compared to the control did not differ (1481 g).

Between 28-35 days, an overall diminution of weight gain for the different treatment groups was observed compared to the control (2217, 2181, 2226, 2211, 2126, 2172, 2182, 2170 g respectively), but none less than the control group.

### Feed Conversion Ratio (Table 10 & Table 12)

Between 0-14 days, FCR of all treatment groups was either the same or worse compared to the control, presumably due to the gastro-intestinal adaptation of the birds to the supplemented diet.

TABLE 12
Feed Conversion Ratios (g feed/g gain) at 14, 21, 28 and 35 days

| Treatment | Day 14 | Day 21 | Day 28 | Day 35 |
|---|---|---|---|---|
| 1 | 1.168 | 1.290 | 1.504 | 1.677 |
| 2 | 1.191 | 1.289 | 1.503 | 1.619 |
| 3 | 1.138 | 1.290 | 1.505 | 1.637 |
| 4 | 1.183 | 1.293 | 1.497 | 1.637 |
| 5 | 1.200 | 1.365 | 1.545 | 1.711 |
| 6 | 1.140 | 1.327 | 1.500 | 1.669 |
| 7 | 1.212 | 1.295 | 1.476 | 1.659 |
| Control | 1.153 | 1.329 | 1.512 | 1.672 |

Values are the means of 4 replicates per treatment

Between 14-21 days, a significant evolution and efficiency in the treatment groups 1, 2, 3, 4, 6, 7 was observed, compared to the control group (1.290, 1.289, 1.293, 1.365, 1.327, 1.295, 1.329 g feed/g gain respectively).

Between 21-28 days, all of the treatment groups (exc. treatment 5 with 1545 g feed/g gain), the FCR maintained the previously observed levels and is better than the control group (1.504, 1.503, 1.505, 1.497, 1.500, 1.476, 1.512 g feed/g gain).

Between 28-35 days, treatments 2, 3, 4 the observed FCR was superior to control (1.619, 1.637, 1.637, 1.672 g feed/g gain respectively).

### CONCLUSIONS

The study was designed to evaluate the response of broilers fed on high standard basal feed formulae to a diet enriched with NovoMax™—a novel biologic compound.

In general, until day 21 all the treatments showed a constant improvement of weight and FCR performance, compared to the control, independent of dose or coating.

Results show that NovoMax™ improves the nutrient absorption in the intestinal tract and the overall metabolic process in the intestinal mucosa (weight and FCR).

1-47. (canceled)

48. A method for preparing an encapsulated proteinaceous bioactive compound in a nutritional food or feed formula, the method comprising:
a. mixing the bioactive compound with a first encapsulating material forming a blend,
b. processing the blend formed in (a) to form a functionally multilayered protected dry blend, wherein the processing of said blend comprises:
(i) forming of a core;
(ii) drying the core formed in step (i);
(iii) collecting the dehydrated core formed in step (ii);
(iv) coating the dehydrated core formed in step (iii) with a liquid comprising a second encapsulating material;
(v) drying the coated core obtained in step (iv) in a fluidized bed and collecting the dehydrated suspension;
(vi) resuspending the dehydrated suspension obtained in step (v) in a liquid comprising a third encapsulating material;
(vii) drying the resuspension obtained in step (vi) in a fluidized bed forming said multilayered protected dry blend;
wherein each of the protective layers is specifically designed to degrade in response to an environmental stimulus and wherein the steps of dehydration is carried out at a maximum temperature of 50° C. such that the activity of said proteinaceous bioactive compound is substantially maintained; and
c. adding the dry blend formed in (b) to said nutritional food or nutritional feed formula thereby preparing a multilayered encapsulated bioactive compound in a nutritional food or nutritional feed formula.

49. The method of claim 48, wherein addition of the encapsulated bioactive compound to the nutritional food or nutritional feed formula is done during manufacturing of said nutritional food or nutritional feed formula, or the addition into said nutritional food or nutritional feed formula is done prior to consumption.

50. The method of claim 48, wherein each of the first, second and third encapsulating materials is the same or different.

51. The method of claim 50, wherein each of the encapsulating materials is food grade or feed grade or pharma grade material.

52. The method of claim 51, wherein each of the encapsulating materials comprises a compound selected from the group consisting of a polysaccharide, a vitamin, an antioxidant, a protease inhibitor, a growth hormone, an EGF (Epidermal Growth Factor), an insulin-like growth factor, an insulin-like growth factor binding protein, an insulin, an immunoglobulins, a proline-rich polypeptide, a lactoferrin, a protease, a lactalbumin, an interleukin, a lysozyme, a TGFA (Transforming Growth Factor A), a PDGF (Platelet Derived Growth Factor) or any combination thereof.

53. The method of claim 52, wherein the encapsulating material comprises a polysaccharide.

54. The method of claim 53, wherein the polysaccharide is selected from the group consisting of maltodextrin, cellulosics and lactose.

55. The method of claim 54, wherein the encapsulating material is maltodextrin.

56. The method of claim 55, wherein the maltodextrin has a dextrose equivalent (DE) between 2 and 64.

57. The method of claim 48, wherein each of the encapsulating materials is specifically formulated to release the bioactive compound as a response to an environmental stimulus.

58. The method of claim 57, wherein said environmental stimulus is selected from the group consisting of moisture content, time, temperature, pressure, pH, ionic strength or enzymatic activity.

59. The method of claim 48, wherein the external of the protective layers is designed to thermally protect the bioactive compound for no less than 2 minutes at a temperature of no less than 95° C.

60. The method of claim 48, wherein the external of said protective layers is designed to protect said bioactive compound from at least one of proteolytic enzymes and pH of no more than 4.75.

61. The method of claim 48, wherein the bioactive compound is extracted from milk, eggs, animal tissue, harvested from recombinant DNA technology, extracted from plants or synthetically produced.

62. The method of claim 48, wherein the bioactive compound is insulin or a functional derivative thereof.

63. The method of claim 48, wherein the bioactive compound is selected from the group consisting of EGF and an analogue of insulin wherein the analogue of insulin is selected from the group consisting of IGF-I and IGF-2.

64. The method of claim 48, wherein the core is inert.

65. The method of claim 48, further comprising an agglomeration step.

66. The method of claim 65, wherein said agglomeration step results in particle average diameter between about 0.1 and about 5,000 micrometers (μm).
